(19) United States
(12) Patent Application Publication
(10) Pub. No.: US 2015/0313503 A1
(43) Pub. Date: Nov. 5, 2015

(54) ELECTROMAGNETIC SENSOR INTEGRATION WITH ULTRATHIN SCANNING FIBER ENDOSCOPE

(71) Applicant: UNIVERSITY OF WASHINGTON THROUGH ITS CENTER FOR COMMERCIALIZATION, Seattle, WA (US)

(72) Inventors: Eric J. Seibel, Seattle, WA (US); David R. HAYNOR, Seattle, WA (US); Timothy D. SOPER, Seattle, WA (US)

(21) Appl. No.: 14/646,209

(22) PCT Filed: Nov. 19, 2013
(86) PCT No.: PCT/US13/70805

§ 371 Date: May 20, 2015

Related U.S. Application Data
(60) Provisional application No. 61/728,410, filed on Nov. 20, 2012.

(51) Int. Cl. A61B 5/06; A61B 1/005; A61B 1/00

(52) U.S. Cl. CPC A61B 5/065; A61B 1/00165; A61B 1/00004; A61B 1/005

(57) ABSTRACT

Methods and systems for imaging internal tissues within a body are provided. In one aspect, a method for imaging an internal tissue of a body is provided. The method includes inserting an image gathering portion of a flexible endoscope into the body. The image gathering portion is coupled to a sensor configured to sense motion of the image gathering portion with respect to fewer than six degrees of freedom. A tracking signal indicative of motion of the image gathering portion is generated using the sensor. The tracking signal is processed in conjunction with supplemental data of motion of the image gathering portion to determine a spatial disposition of the image gathering portion within the body. In many embodiments, the method includes collecting a tissue sample from the internal tissue.

### CROSS-REFERENCE

This application claims the benefit of U.S. Provisional Application No. 61/728,410 filed Nov. 20, 2012, which application is incorporated herein by reference.

### STATEMENT AS TO FEDERALLY SPONSORED RESEARCH

This invention was made with government support under CA094303 awarded by the National Institutes of Health. The government may have certain rights in the invention.

### BACKGROUND

A definitive diagnosis of lung cancer typically requires a biopsy of potentially cancerous lesions identified through high-resolution computer tomography (CT) scanning Various techniques can be used to collect a tissue sample from within the lung. For example, transbronchial biopsy (TBB) typically involves inserting a flexible bronchoscope into the patient's lung through the trachea and central airways, followed by advancing a biopsy tool through a working channel of the bronchoscope to access the biopsy site. As TBB is safe and minimally invasive, it is frequently preferred over more invasive procedures such as transthoracic needle biopsy.

Current systems and methods for TBB, however, can be less than ideal. For example, the relatively large diameter of current bronchoscopes (5-6 mm) precludes insertion into small airways of the peripheral lung where lesions are commonly found. In such instances, clinicians may be forced to perform blind biopsies in which the biopsy tool is extended outside the field of view of the bronchoscope, thus reducing the accuracy and diagnostic yield of TBB. Additionally, current TBB techniques utilizing fluoroscopy to aid the navigation of the bronchoscope and biopsy tool within the lung can be costly and inaccurate, and pose risks to patient safety in terms of radiation exposure. Furthermore, such fluoroscopic images are typically two-dimensional (2D) images, which can be less than ideal for visual navigation within a three-dimensional (3D) environment.

Thus, there is a need for improved methods and systems for imaging internal tissues within a patient's body, such as within a peripheral airway of the lung.

### SUMMARY

Methods and systems for imaging internal tissues within a body are provided. For example, in many embodiments, the methods and systems described herein provide tracking of an image gathering portion of an endoscope. In many embodiments, a tracking signal is generated by a sensor coupled to the image gathering portion and configured to track motion with respect to fewer than six degrees of freedom (DoF). The tracking signal can be processed in conjunction with supplemental motion data (e.g., motion data from a second tracking sensor or image data from the endoscope) to determine the 3D spatial disposition of the image gathering portion of the endoscope within the body. The method and systems described herein are suitable for use with ultrathin endoscopic systems, thus enabling imaging of tissues within narrow lumens and/or small spaces within the body. Additionally, in many embodiments, the disclosed methods and systems can be used to generate 3D virtual models of internal structures of the body, thereby providing improved navigation to a surgical site.

Thus, in one aspect, a method for imaging an internal tissue of a body is provided. The method includes inserting an image gathering portion of a flexible endoscope into the body. The image gathering portion is coupled to a sensor configured to sense motion of the image gathering portion with respect to fewer than six degrees of freedom. A tracking signal indicative of motion of the image gathering portion is generated using the sensor. The tracking signal is processed in conjunction with supplemental data of motion of the image gathering portion to determine a spatial disposition of the image gathering portion within the body. In many embodiments, the method includes collecting a tissue sample from the internal tissue.

In many embodiments, the sensor is configured to sense motion of the image gathering portion with respect to five degrees of freedom. The sensor can include an electromagnetic tracking sensor. The electromagnetic tracking sensor can include an annular sensor disposed around the image gathering portion.

In many embodiments, the supplemental data includes a second tracking signal indicative of motion of the image gathering portion generated by a second sensor configured to sense motion of the image gathering portion with respect to fewer than six degrees of freedom. For example, the second sensor can be configured to sense motion of the image gathering portion with respect to five degrees of freedom. The sensor and the second sensor each can include an electromagnetic sensor.

In many embodiments, the supplemental data includes one or more images collected by the image gathering portion. The supplemental data can further include a virtual model of the body to which the one or more images can be registered.

In many embodiments, processing the tracking signal in conjunction with supplemental data of motion of the image gathering portion to determine a spatial disposition of the image gathering portion within the body includes adjusting for tracking errors caused by motion of the body due to a body function.

In another aspect, a system is provided for imaging an internal tissue of a body. The system includes a flexible endoscope including an image gathering portion and a sensor coupled to the image gathering portion. The sensor is configured to generate a tracking signal indicative of motion of the image gathering portion with respect to fewer than six degrees of freedom. The system includes one or more processors and a tangible storage medium storing non-transitory instructions that, when executed by the one or more processors, process the tracking signal in conjunction with supplemental data of motion of the image gathering portion to determine a spatial disposition of the image gathering portion within the body.

In many embodiments, the image gathering portion includes a cantilevered optical fiber configured to scan light onto the internal tissue and a light sensor configured to receive light returning from the internal tissue so as to generate an output signal that can be processed to provide images of the internal tissue. The diameter of the image gathering portion can be less than or equal to 2 mm, less than or equal to 1.6 mm, or less than or equal to 1.1 mm.

In many embodiments, the flexible endoscope includes a steering mechanism configured to guide the image gathering portion within the body.

In many embodiments, the sensor is configured to sense motion of the image gathering portion with respect to five degrees of freedom. The sensor can include an electromagnetic tracking sensor. The electromagnetic tracking sensor can include an annular sensor disposed around the image gathering portion.

In many embodiments, a second sensor is coupled to the image gathering portion and configured to generate a second tracking signal indicative of motion of the image gathering portion with respect to fewer than six degrees of freedom, such that the supplemental data of motion includes the second tracking signal. The second sensor can be configured to sense motion of the image gathering portion with respect to five degrees of freedom. The sensor and the second sensor can each include an electromagnetic tracking sensor.

In many embodiments, the supplemental motion data includes one or more images collected by the image gathering portion. The supplemental data can further include a virtual model of the body to which the one or more images can be registered.

In many embodiments, the tangible storage medium stores non-transitory instructions that, when executed by the one or more processors, process the tracking signal in conjunction with the supplemental data of motion of the image gathering portion to determine a spatial disposition of the image gathering portion within the body while adjusting for tracking errors caused by motion of the body due to a body function.

In another aspect, a method for generating a virtual model of an internal structure of the body is provided. The method includes generating first image data of an internal structure of a body with respect to a first camera viewpoint and generating second image data of the internal structure with respect to a second camera viewpoint, the second camera viewpoint being different than the first camera viewpoint. The first image data and the second image data can be processed to generate a virtual model of the internal structure.

In many embodiments, a second virtual model of a second internal structure of the body can be registered with the virtual model of the internal structure. The second internal structure can include subsurface features relative to the internal structure. The second virtual model can be generated via one or more of: (a) a computed tomography scan, (b) magnetic resonance imaging, (c) positron emission tomography, (d) fluoroscopic imaging, and (e) ultrasound imaging.

In many embodiments, the first and second image data are generated using one or more endoscopes each having an image gathering portion. The first and second image data can be generated using a single endoscope. The one or more endoscopes can include at least one rigid endoscope, the rigid endoscope having a proximal end extending outside the body. A spatial disposition of an image gathering portion of the rigid endoscope relative to the internal structure can be determined by tracking a spatial disposition of the proximal end of the rigid endoscope.

In many embodiments, each image gathering portion of the one or more endoscopes can be coupled to a sensor configured to sense motion of the image gathering portion with respect to fewer than six degrees of freedom to generate a tracking signal indicative of the motion. The tracking signal can be processed in conjunction with supplemental data of motion of the image gathering portion to determine first and second spatial dispositions relative to the internal structure. The sensor can include an electromagnetic sensor.

In many embodiments, each image gathering portion of the one or more endoscopes includes a second sensor configured to sense motion of the image gathering portion with respect to fewer than six degrees of freedom to generate a second tracking signal indicative of motion of the image gathering portion, such that the supplemental data includes the second tracking signal. The sensor and the second sensor can each include an electromagnetic tracking sensor. The supplemental data can include image data generated by the image gathering portion.

In another aspect, a system for generating a virtual model of an internal structure of a body is provided. The system includes one or more endoscopes, each including an image gathering portion. The system includes one or more processors and a tangible storage medium storing non-transitory instructions that, when executed by the one or more processors, process first image data of an internal structure of a body and second image data of the internal structure to generate a virtual model of the internal structure. The first image data is generated using an image gathering portion of the one or more endoscopes in a first spatial disposition relative to the internal structure. The second image data is generated using an image gathering portion of the one or more endoscopes in a second spatial disposition relative to the internal structure, the second spatial disposition being different from the first spatial disposition.

In many embodiments, the one or more endoscopes consists of a single endoscope. At least one image gathering portion of the one or more endoscopes can include a cantilevered optical fiber configured to scan light onto the internal tissue and a light sensor configured to receive light returning from the internal tissue so as to generate an output signal that can be processed to provide images of the internal tissue.

In many embodiments, the tangible storage medium stores non-transitory instructions that, when executed by the one or more processors, registers a second virtual model of a second internal structure of the body with the virtual model of the internal structure. The second virtual model can be generated via an imaging modality other than the one or more endoscopes. The second internal structure can include subsurface features relative to the internal structure. The imaging modality can include one or more of (a) a computed tomography scan, (b) magnetic resonance imaging, (c) positron emission tomography, (d) fluoroscopic imaging, and/or (e) ultrasound imaging.

In many embodiments, at least one of the one or more endoscopes is a rigid endoscope, the rigid endoscope having a proximal end extending outside the body. A spatial disposition of an image gathering portion of the rigid endoscope relative to the internal structure can be determined by tracking a spatial disposition of the proximal end of the rigid endoscope.

In many embodiments, a sensor is coupled to at least one image gathering portion of the one or more endoscopes and configured to sense motion of the image gathering portion with respect to fewer than six degrees of freedom to generate a tracking signal indicative of the motion. The tracking signal can be processed in conjunction with supplemental data of motion of the image gathering portion to determine a spatial disposition of the image gathering portion relative to the internal structure. The sensor can include an electromagnetic tracking sensor. The system can include a second sensor configured to sense motion of the image gathering portion with respect to fewer than six degrees of freedom to generate a second tracking signal indicative of motion of the image gathering portion, such that the supplemental data includes the second tracking signal. The sensor and the second sensor each can include an electromagnetic sensor. The supplemental data can include image data generated by the image gathering portion.

Other objects and features of the present invention will become apparent by a review of the specification, claims, and appended figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a flexible endoscope system, in accordance with many embodiments;

FIG. 1B shows a cross-section of the distal end of the flexible endoscope of FIG. 1A, in accordance with many embodiments;

FIGS. 2A and 2B illustrate a biopsy tool suitable for use within ultrathin endoscopes, in accordance with many embodiments;

FIG. 3 illustrates an electromagnetic tracking (EMT) system for tracking an endoscope within the body of a patient, in accordance with many embodiments;

FIG. 4A illustrates the distal portion of an ultrathin endoscope with integrated EMT sensors, in accordance with many embodiments;

FIG. 4B illustrates the distal portion of an ultrathin scanning fiber endoscope with an annular EMT sensor, in accordance with many embodiments;

FIG. 5 is a block diagram illustrating acts of a method for tracking a flexible endoscope within the body in accordance with many embodiments;

FIG. 6A illustrates a scanning fiber bronchoscope (SFB) compared to a conventional bronchoscope, in accordance with many embodiments;

FIG. 6B illustrates calibration of a SFB having a coupled EMT sensor, in accordance with many embodiments;

FIG. 6C illustrates registration of EMT system and computed tomography (CT) generated image coordinates, in accordance with many embodiments;

FIG. 6D illustrates EMT sensors placed on the abdomen and sternum to monitor respiration, in accordance with many embodiments;

FIG. 7A illustrates correction of radial lens distortion of an image, in accordance with many embodiments;

FIG. 7B illustrates conversion of a color image to grayscale, in accordance with many embodiments;

FIG. 7C illustrates vignetting compensation of an image, in accordance with many embodiments;

FIG. 7D illustrates noise removal from an image, in accordance with many embodiments;

FIG. 8A illustrates a 2D input video frame, in accordance with many embodiments;

FIGS. 8B and 8C are vector images defining p and q gradients, respectively, in accordance with many embodiments;

FIG. 8D illustrates a virtual bronchoscopic view obtained from the CT-based reconstruction, in accordance with many embodiments;

FIGS. 8E and 8F are vector images illustrating surface gradients p′ and q′, respectively, in accordance with many embodiments;

FIG. 9A illustrates variation of δ and θ with time, in accordance with many embodiments;

FIG. 9B illustrates respiratory motion compensation (RMC), in accordance with many embodiments;

FIG. 9C is a schematic illustration by way of block diagram illustrating a hybrid tracking algorithm, in accordance with many embodiments;

FIG. 10 illustrates tracked position and orientation of the SFB using electromagnetic tracking (EMT) and image-based tracking (IBT), in accordance with many embodiments;

FIG. 11 illustrating tracking results from a bronchoscopy session, in accordance with many embodiments;

FIG. 12 illustrates tracking accuracy of tracking methods from a bronchoscopy session, in accordance with many embodiments;

FIG. 13 illustrates z-axis tracking results for hybrid methods within a peripheral region, in accordance with many embodiments;

FIG. 14 illustrates registered real and virtual bronchoscopic views, in accordance with many embodiments;

FIG. 15 illustrates a comparison of the maximum deformation approximated by a Kalman filter to that calculated from the deformation field, in accordance with many embodiments;

FIG. 16 illustrates an endoscopic system, in accordance with many embodiments;

FIG. 17 illustrates another endoscopic system, in accordance with many embodiments;

FIG. 18 illustrates yet another endoscopic system, in accordance with many embodiments; and

FIG. 19 is a block diagram illustrating acts of a method for generating a virtual model of an internal structure of a body, in accordance with many embodiments.

### DETAILED DESCRIPTION

Methods and systems are described herein for imaging internal tissues within a body (e.g., bronchial passages within the lung). In many embodiments, the methods and systems disclosed provide tracking of an image gathering portion of an endoscope within the body using a coupled sensor measuring motion of the image gathering portion with respect to less than six DoF. The tracking data measured by the sensor can be processed in conjunction with supplemental motion data (e.g., tracking data provided by a second sensor and/or images from the endoscope) to determine the full motion of the image gathering portion (e.g., with respect to six DoF: three DoF in translation and three DoF in rotation) and thereby determine the 3D spatial disposition of the image gathering portion within the body. In many embodiments, the motion sensors described herein (e.g., five DoF sensors) are substantially smaller than current six DoF motion sensors. Accordingly, the disclosed methods and systems enable the development of ultrathin endoscopes that can be tracked within the body with respect to six DoF of motion.

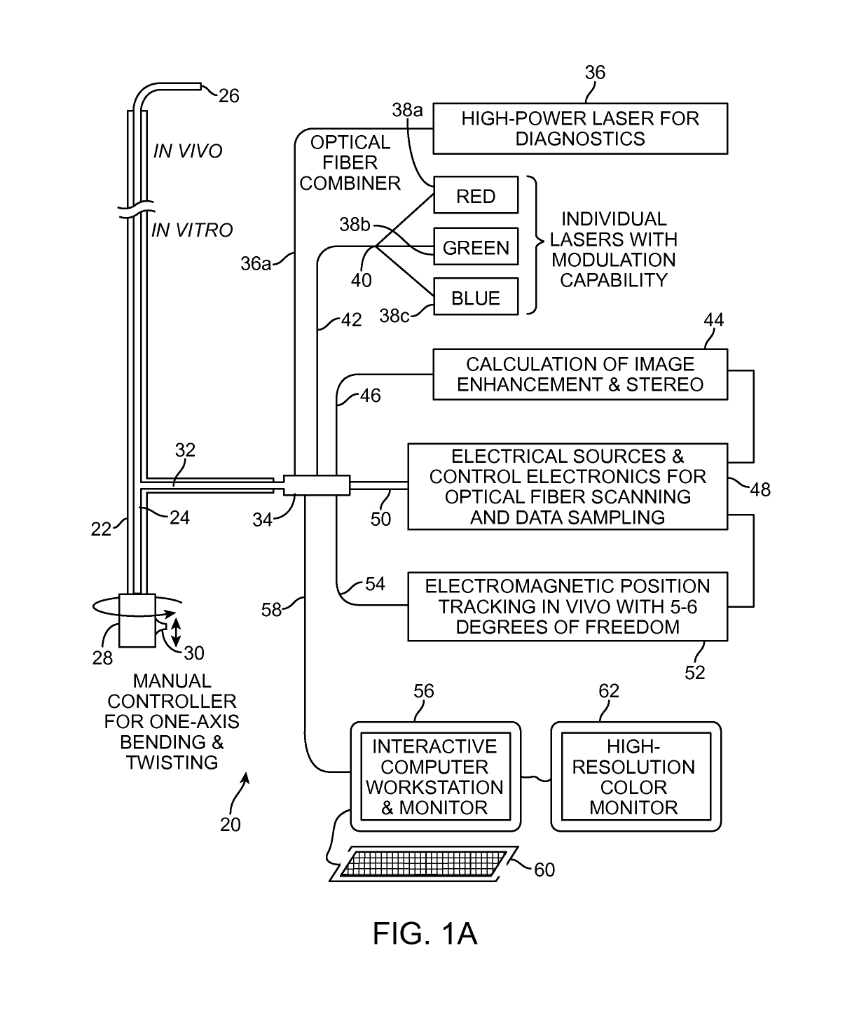

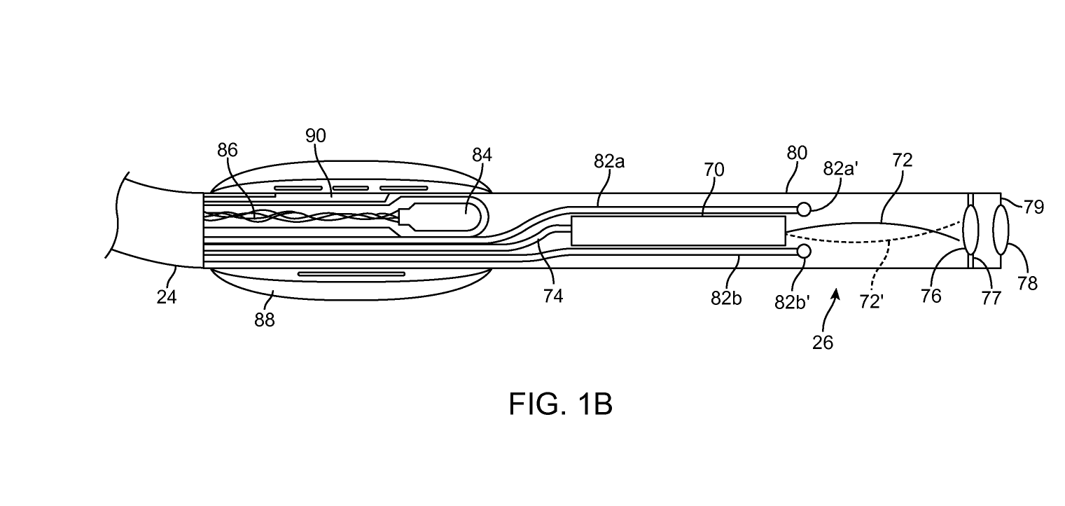

Turning now to the drawings, in which like numbers designate like elements in the various figures, FIG. 1A illustrates a flexible endoscope system 20, in accordance with many embodiments of the present invention. The system 20 includes a flexible endoscope 24 that can be inserted into the body through a multi-function endoscopic catheter 22. The flexible endoscope 24 includes a relatively rigid distal tip 26 housing a scanning optical fiber, described in detail below. The proximal end of the flexible endoscope 24 includes a rotational control 28 and a longitudinal control 30, which respectively rotate and move the flexible endoscope longitudinally relative to catheter 22, providing manual control for one-axis bending and twisting. Optionally, the flexible endoscope 24 can include a steering mechanism (not shown) to guide the distal tip 26 within the body. Various electrical leads and/or optical fibers (not separately shown) extend from the endoscope 24 through a branch arm 32 to a junction box 34.

Light for scanning internal tissues near the distal end of the flexible endoscope can be provided either by a high power laser 36 through an optical fiber 36a, or through optical fibers 42 by individual red (e.g., 635 nm), green (e.g., 532 nm), and blue (e.g., 440 nm) lasers 38a, 38b, and 38c, respectively, each of which can be modulated separately. Colored light from lasers 38a, 38b, and 38c can be combined into a single optical fiber 42 using an optical fiber combiner 40. The light can be directed through the flexible endoscope 24 and emitted from the distal tip 26 to scan adjacent tissues.

A signal corresponding to reflected light from the scanned tissue can either be detected with sensors disposed within and/or near the distal tip 26 or conveyed through optical fibers extending back to junction box 34. This signal can be processed by several modules, including a module 44 for calculating image enhancement and providing stereo imaging of the scanned region. The module 44 can be operatively coupled to junction box 34 through leads 46. Electrical sources and control electronics 48 for optical fiber scanning and data sampling (e.g., from the scanning and imaging unit within distal tip 26) can be coupled to junction box 34 through leads 50. A sensor (not shown) can provide signals that enable tracking of the distal tip 26 of the flexible endoscope 24 in vivo to a tracking module 52 through leads 54. Suitable embodiments of sensors for in vivo tracking are described below.

An interactive computer workstation and monitor 56 with an input device 60 (e.g., a keyboard, a mouse, a touch screen) is coupled to junction box 34 through leads 58. The interactive computer workstation can be connected to a display unit 62 (e.g., a high resolution color monitor) suitable for displaying detailed video images of the internal tissues through which the flexible endoscope 24 is being advanced.

FIG. 1B shows a cross-section of the distal tip 26 of the flexible endoscope 24, in accordance with many embodiments. The distal tip 26 includes a housing 80. An optional balloon 88 can be disposed external to the housing 80 and can be inflated to stabilize the distal tip 26 within a passage of the patient's body. A cantilevered scanning optical fiber 72 is disposed within the housing and is driven by a two-axis piezoelectric driver 70 (e.g., to a second position 72′). In many embodiments, the driver 70 drives the scanning fiber 72 in mechanical resonance to move in a suitable 2D scanning pattern, such as a spiral scanning pattern, to scan light onto an adjacent surface to be imaged (e.g., an internal tissue or structure). Light from an external light source, such as a laser from the system 20, can be conveyed through a single mode optical fiber 74 to the scanning optical fiber 72. The lenses 76 and 78 can focus the light emitted by the scanning optical fiber 72 onto the adjacent surface. Light reflected from the surface can enter the housing 80 through lenses 76 and 78 and/or optically clear windows 77 and 79. The windows 77 and 79 can have optical filtering properties. Optionally, the window 77 can support the lens 76 within the housing 80.

The reflected light can be conveyed through multimode optical return fibers 82a and 82b having respective lenses 82a′ and 82b′ to light detectors disposed in the proximal end of the flexible endoscope 24. Alternatively, the multimode optical return fibers 82a and 82b can be terminated without the lens 82a′ and 82b′. For example, the fibers 82a and 82b can pass through the annular space of the window 77 and terminate in a disposition peripheral to and surrounding the lens 78 within the distal end of the housing 80. In many embodiments, the distal ends of the fibers 82a and 82b can be disposed flush against the window 79 or replace the window 79. Alternatively, the optical return fibers 82a and 82b can be separated from the fiber scan illumination and be included in any suitable biopsy tool that has optical communication with the scanned illumination field. Although FIG. 1B depicts two optical return fibers, any suitable number and arrangement of optical return fibers can be used, as described in further detail below. The light detectors can be disposed in any suitable location within or near the distal tip 26 of the flexible endoscope 24. Signals from the light detectors can be conveyed to processing modules external to the body (e.g., via junction box 34) and processed to provide a video image of the internal tissue or structure to the user (e.g., on display unit 62).

In many embodiments, the flexible endoscope 24 includes a sensor 84 that produces signals indicative of the position and/or orientation of the distal tip 26 of the flexible endoscope. While FIG. 1B depicts a single sensor disposed within the proximal end of the housing 80, many configurations and combinations of suitable sensors can be used, as described below. The signals produced by the sensor 84 can be conveyed through electrical leads 86 to a suitable memory unit and processing unit, such as memory and processors within the interactive computer workstation and monitor 56, to produce tracking data indicative of the 3D spatial disposition of the distal tip 26 within the body.

The tracking data can be displayed to the user, for example, on display unit 62. In many embodiments, the displayed tracking data can be used to guide the endoscope to an internal tissue or structure of interest within the body (e.g., a biopsy site within the peripheral airways of the lung). For example, the tracking data can be processed to determine the spatial disposition of the endoscope relative to a virtual model of the surgical site or body cavity (e.g., a virtual model created from a high-resolution computed tomography (CT) scan, magnetic resonance imaging (MRI), positron emission tomography (PET), fluoroscopic imaging, and/or ultrasound imaging). The real-time location and orientation of the endoscope within the virtual model can thus be displayed to a clinician during an endoscopic procedure. In many embodiments, the display unit 62 can also display a path (e.g., overlaid with the virtual model) along which the endoscope can be navigated to reach a specified target site within the body. Consequently, additional visual guidance can be provided by comparing the current spatial disposition of the endoscope relative to the path.

In many embodiments, the flexible endoscope 24 is an ultrathin flexible endoscope having dimensions suitable for insertion into small diameter passages within the body. In many embodiments, the housing 80 of the distal tip 26 of the flexible endoscope 24 can have an outer diameter of 2 mm or less, 1.6 mm or less, or 1.1 mm or less. This size range can be applied, for example, to bronchoscopic examination of eighth to tenth generation bronchial passages.

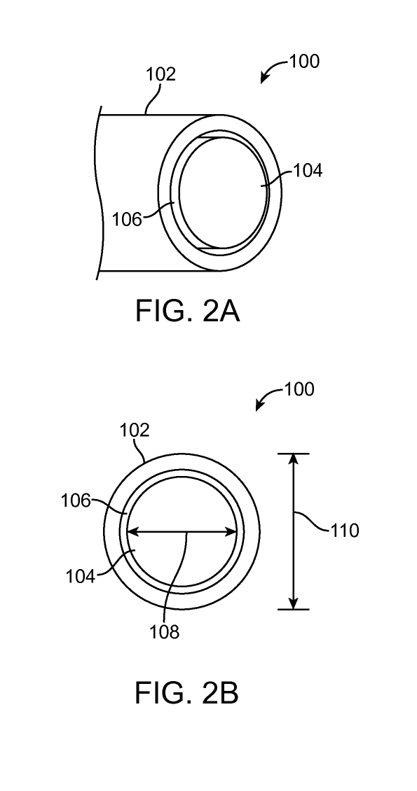

FIGS. 2A and 2B illustrate a biopsy tool 100 suitable for use with ultrathin endoscopes, in accordance with many embodiments. The biopsy tool 100 includes a cannula 102 configured to fit around the image gathering portion 104 of an ultrathin endoscope. In many embodiments, a passage 106 is formed between the cannula 102 and image gathering portion 104. The image gathering portion 104 can have any suitable outer diameter 108, such as a diameter of 2 mm or less, 1.6 mm or less, or 1.1 mm or less. The cannula can have any outer diameter 110 suitable for use with an ultrathin endoscope, such as a diameter of 2.5 mm or less, 2 mm or less, or 1.5 mm or less. The biopsy tool 100 can be any suitable tool for collecting cell or tissue samples from the body. For example, a biopsy sample can be aspirated into the passage 106 of the cannula 102 (e.g., via a lavage or saline flush technique). Alternatively or in combination, the exterior lateral surface of the cannula 102 can include a tubular cytology brush or scraper. Optionally, the cannula 102 can be configured as a sharpened tube, helical cutting tool, or hollow biopsy needle. The embodiments described herein advantageously enable biopsying of tissues with guidance from ultrathin endoscopic imaging.

Electromagnetic Tracking

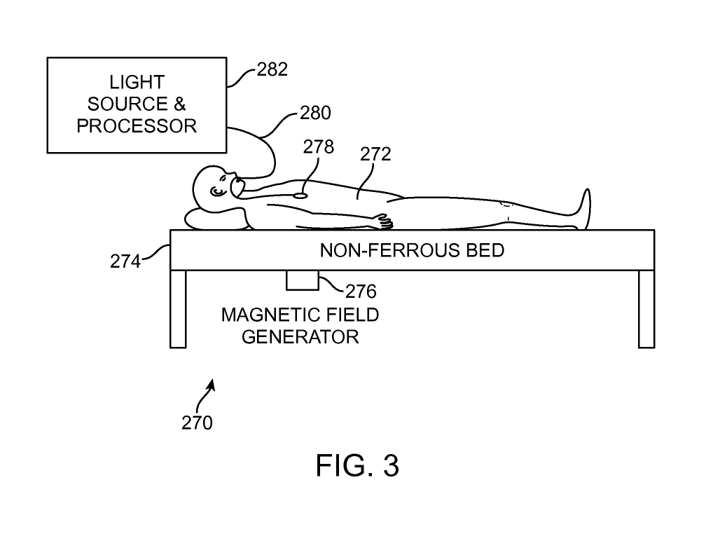

FIG. 3 illustrates an electromagnetic tracking (EMT) system 270 for tracking an endoscope within the body of a patient 272, in accordance with many embodiments. The system 270 can be combined with any suitable endoscope and any suitable EMT sensor, such as the embodiments described herein. In the system 270, a flexible endoscope is inserted within the body of a patient 272 lying on a non-ferrous bed 274. An external electromagnetic field transmitter 276 produces an electromagnetic field penetrating the patient's body. An EMT sensor 278 can be coupled to the distal end of the endoscope and can respond to the electromagnetic field by producing tracking signals indicative of the position and/or orientation of the distal end of the flexible endoscope relative to the transmitter 276. The tracking signals can be conveyed through a lead 280 to a processor within a light source and processor 282, thereby enabling real-time tracking of the distal end of the flexible endoscope within the body.

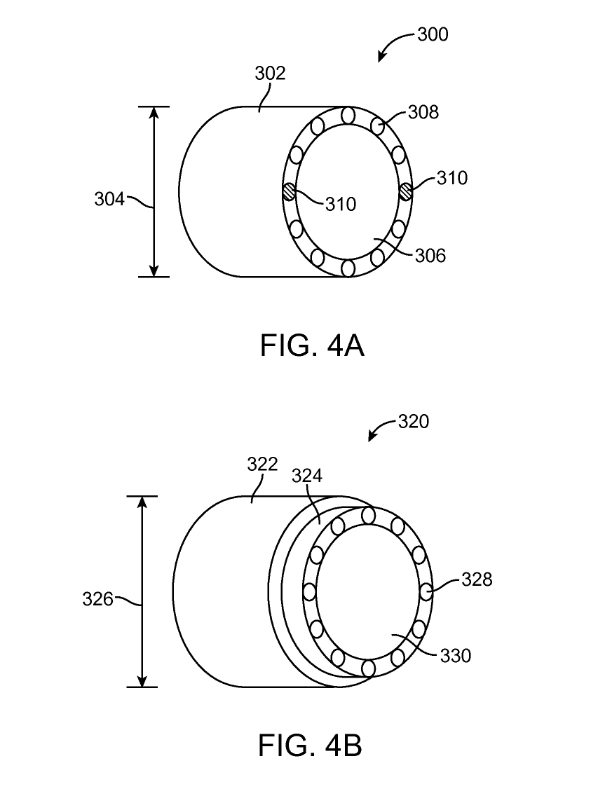

FIG. 4A illustrates the distal portion of an ultrathin scanning fiber endoscope 300 with integrated EMT sensors, in accordance with many embodiments. The scanning fiber endoscope 300 includes a housing or sheath 302 having an outer diameter 304. For example, the outer diameter 304 can be 2 mm or less, 1.6 mm or less, or 1.1 mm or less. A scanning optical fiber unit (not shown) is disposed within the lumen 306 of the sheath 302. Optical return fibers 308 and EMT sensors 310 can be integrated into the sheath 302. Alternatively or in combination, one or more EMT sensors 310 can be coupled to the exterior of the sheath 302 or affixed within the lumen 306 of the sheath 302. The optical return fibers 308 can capture and convey reflected light from the surface being imaged. Any suitable number of optical return fibers can be used. For example, the ultrathin endoscope 300 can include at least six optical return fibers. The optical fibers can be made of any suitable light transmissive material (e.g., plastic or glass) and can have any suitable diameter (e.g., approximately 0.25 mm).

The EMT sensors 310 can provide tracking signals indicative of the motion of the distal portion of the ultrathin endoscope 300. In many embodiments, each of the EMT sensors 310 provides tracking with respect to fewer than six DoF of motion. Such sensors can advantageously be fabricated in a size range suitable for integration with embodiments of the ultrathin endoscopes described herein. For example, EMT sensors tracking the motion of the distal portion with respect to five DoF (e.g., excluding longitudinal rotation) can be manufactured with a diameter of 0.3 mm or less.

Any suitable number of EMT sensors can be used. For example, the ultrathin endoscope 300 can include two five DoF EMT sensors configured such that the missing DoF of motion of the distal portion can be recovered based on the differential spatial disposition of the two sensors. Alternatively, the ultrathin endoscope 300 can include a single five DoF EMT sensor, and the roll angle can be recovered by combining the tracking signal from the sensor with supplemental data of motion, as described below.

FIG. 4B illustrates the distal portion of an ultrathin scanning fiber endoscope 320 with an annular EMT sensor 322, in accordance with many embodiments. The annular EMT sensor 322 can be disposed around the sheath 324 of the ultrathin endoscope 300 and has an outer diameter 326. The outer diameter 326 of the annular sensor 322 can be any size suitable for integration with an ultrathin endoscope, such as 2 mm or less, 1.6 mm or less, or 1.1 mm or less. A plurality of optical return fibers 328 can be integrated into the sheath 324. A scanning optical fiber unit (not shown) is disposed within the lumen 330 of the sheath 324. Although FIG. 4B depicts the annular EMT sensor 322 as surrounding the sheath 324, other configurations of the annular sensor 322 are also possible. For example, the annular sensor 322 can be integrated into the sheath 324 or affixed within the lumen 330 of the sheath 324. Alternatively, the annular sensor 322 can be integrated into a sheath or housing of a device configured to fit over the sheath 324 for use with the scanning fiber endoscope 320, such as the cannula of a biopsy tool as described herein.

In many embodiments, the annular EMT sensor 322 can be fixed to the sheath 324 such that the sensor 322 and the sheath 324 move together. Accordingly, the annular EMT sensor 322 can provide tracking signals indicative of the motion of the distal portion of the ultrathin endoscope 320. In many embodiments, the annular EMT sensor 322 tracks motion with respect to fewer than six DoF. For example, the annular EMT sensor 322 can provide tracking with respect to five DoF (e.g., excluding the roll angle). The missing DoF can be recovered by combining the tracking signal from the sensor 322 with supplemental data of motion. In many embodiments, the supplemental data of motion can include a tracking signal from at least one other EMT sensor measuring less than six DoF of motion of the distal portion, such that the missing DoFs can be recovered based on the differential spatial disposition of the sensors. For example, similar to the embodiment of FIG. 4A, one or more of the optical return fibers 328 can be replaced with a five DoF EMT sensor.

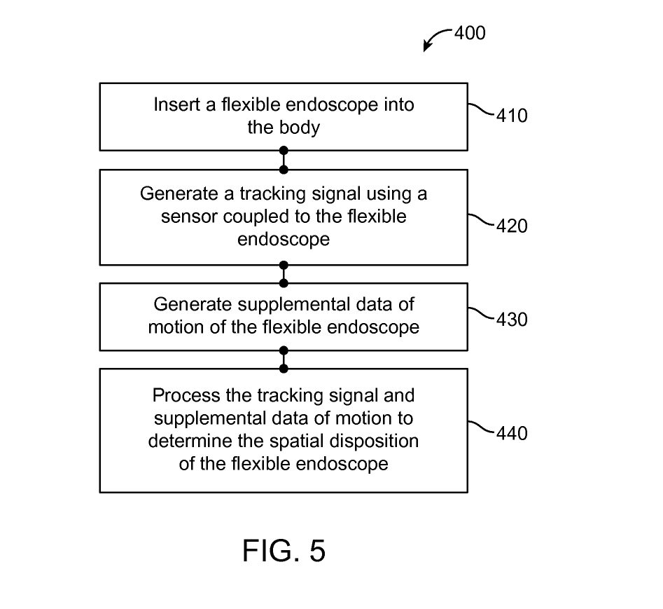

FIG. 5 is a block diagram illustrating acts of a method 400 for tracking a flexible endoscope within the body, in accordance with many embodiments of the present invention. Any suitable system or device can be used to practice the method 400, such the embodiments described herein.

In act 410, a flexible endoscope is inserted into the body of a patient. The endoscope can be inserted via a surgical incision suitable for minimally invasive surgical procedures. Alternatively, the endoscope can be inserted into a natural body opening. For example, the distal end of the endoscope can be inserted into and advanced through an airway of the lung for a bronchoscopic procedure. Any suitable endoscope can be used, such as the embodiments described herein.

In act 420, a tracking signal is generated by using a sensor coupled to the flexible endoscope (e.g., coupled to the image gathering portion at the distal end of the endoscope). Any suitable sensor can be used, such as the embodiments of FIGS. 4A and 4B. In many embodiments, each sensor provides a tracking signal indicative of the motion of the endoscope with respect to fewer than six DoF, as described herein.

In act 430, supplemental data of motion of the flexible endoscope is generated. The supplemental motion data can be processed in conjunction with the tracking signal to determine the spatial disposition of the flexible endoscope with respect to six DoF. For example, the supplemental motion data can include a tracking signal obtained from a second EMT sensor tracking motion with respect to fewer than six DoF, as previously described in relation to FIGS. 4A and 4B. Alternatively or in combination, the supplemental data of motion can include a tracking signal produced in response to an electromagnetic tracking field produced by a second electromagnetic transmitter, and the missing DoF can be recovered by comparing the spatial disposition of the sensor relative to the two reference frames defined by the transmitters.

Alternatively or in combination, the supplemental data of motion can include image data that can be processed to recover the DoF of motion missing from the EMT sensor data (e.g., the roll angle). In many embodiments, the image data includes image data collected by the endoscope. Any suitable ego-motion estimation technique can be used to recover the missing DoF of motion from the image data, such as optical flow or camera tracking. For example, successive images captured by the endoscope can be compared and analyzed to determine the spatial transformation of the endoscope between images.

Alternatively or in combination, the spatial disposition of the endoscope can be estimated using image data collected by the endoscope and a 3D virtual model of the body (hereinafter “image-based tracking” or “IBT”). IBT can be used to determine the position and orientation of the endoscope with respect to up to six DoF. For example, a series of endoscopic images can be registered to a 3D virtual model of the body (e.g., generated from prior scan data obtained through obtained through CT, MRI, PET, fluoroscopy, ultrasound, and/or any other suitable imaging modality). For each image or frame, a spatial disposition of a virtual camera within the virtual model can be determined that maximizes the similarity between the image and a virtual image taken from the viewpoint of the virtual camera. Accordingly, the motion of the camera used to produce the corresponding image data can be reconstructed with respect to up to six DoF.

In act 440, the tracking signal and the supplemental data of motion are processed to determine the spatial disposition of the flexible endoscope within the body. Any suitable device can be used to perform the act 440, such as the workstation 56 or tracking module 52. For example, the workstation 56 can include a tangible computer-readable storage medium storing suitable non-transitory instructions that can be executed by one or more processors of the workstation 56 to process the tracking signal and the supplemental data. The spatial disposition information can be presented to the user on a suitable display unit to aid in endoscope navigation, as previously described herein. For example, the spatial disposition of the flexible endoscope can displayed along with one or more of a virtual model of the body (e.g., generated as described above), a predetermined path of the endoscope, and real-time image data collected by the endoscope.

Hybrid Tracking

In many embodiments, a hybrid tracking approach combining EMT data and IBT data can be used to track an endoscope within the body. Advantageously, the hybrid tracking approach can combine the stability of EMT data and accuracy of IBT data while minimizing the influence of measurement errors from a single tracking system. Furthermore, in many embodiments, the hybrid tracking approach can be used to determine the spatial disposition of the endoscope within the body while adjusting for tracking errors caused by motion of the body, such as motion due to a body function (e.g., respiration). The hybrid tracking approach can be performed with any suitable embodiment of the systems, methods, and devices described herein. For example, the hybrid tracking approach can be used to calculate the six-dimensional (6D) position and orientation, \tilde{x}= [symbol] x, y, z, θ, φ, y [symbol] , of an ultrathin scanning fiber bronchoscope (SFB) with a coupled EMT sensor as previously described.

Although the following embodiments are described in terms of bronchoscopy, the hybrid tracking approaches described herein can be applied to any suitable endoscopic procedure. Additionally, although the following embodiments are described with regards to endoscope tracking within a pig, the hybrid tracking approaches described herein can be applied to any suitable human or animal subject. Furthermore, although the following embodiments are described in terms of a tracking simulation, the hybrid tracking approaches described herein can be applied to real-time tracking during an endoscopic procedure.

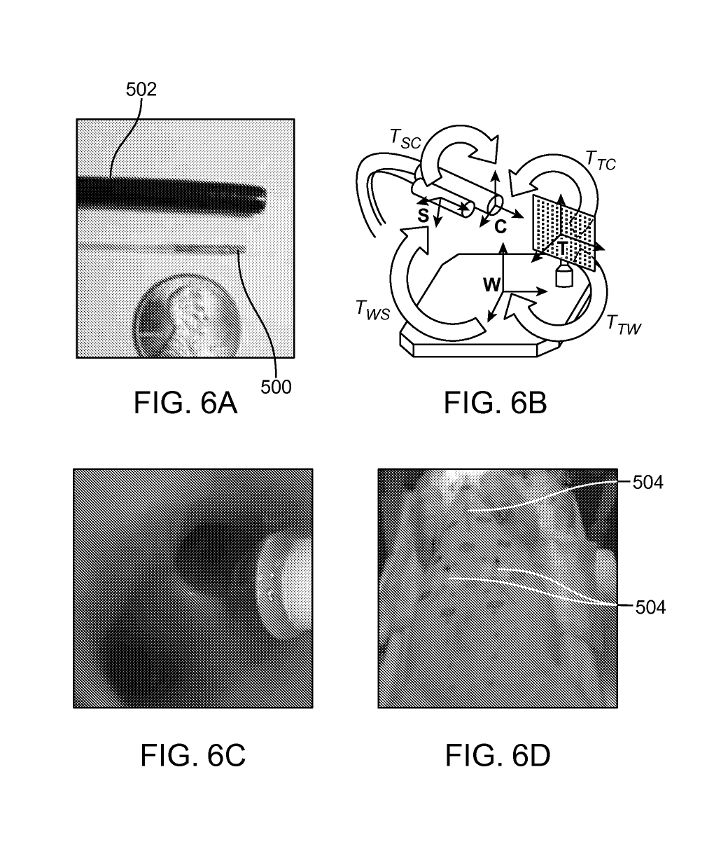

Any suitable endoscope and sensing system can be used for the hybrid tracking approaches described herein. For example, an ultrathin (1.6 mm outer diameter) single SFB capable of high-resolution (500×500), full-color, video rate (30 Hz) imaging can be used. FIG. 6A illustrates a SFB 500 compared to a conventional bronchoscope 502, in accordance with many embodiments. A custom hybrid system can be used for tracking the SFB in peripheral airways using an EMT system and miniature sensor (e.g., manufactured by Ascension Technology Corporation) and IBT of the SFB video with a preoperative CT. In many embodiments, a Kalman filter is employed to adaptively estimate the positional and orientational error between the two tracking inputs. Furthermore, a means of compensating for respiratory motion can include intraoperatively estimating the local deformation at each video frame. The hybrid tracking model can be evaluated, for example, by using it for in vivo navigation within a live pig.

Animal Preparation

A pig was anesthesized for the duration of the experiment by continuous infusion. Following tracheotomy, the animal was intubated and placed on a ventilator at a rate of 22 breaths/min and a volume of 10 mL/kg. Subsequent bronchoscopy and CT imaging of the animal was performed in accordance with a protocol approved by the University of Washington Animal Care Committee.

Free-Hand System Calibration

Prior to bronchoscopy, a miniature EMT sensor can be attached to the distal tip of the SFB using a thin section of silastic tubing. A free-hand system calibration can then be conducted to relate the 2D pixel space of the video images produced by the SFB to that of the 3D operative environment, with respect to coordinate systems of the world (W), sensor (S), camera (C), and test target (T). Based on the calibration, transformations T_{SC}, T_{TC}, T_{WS}, and T_{TW} can be computed between pairs of coordinate systems (denoted by the subscripts). FIG. 6B illustrates calibration of a SFB having a coupled EMT sensor, in accordance with many embodiments. For example, the test target can be imaged from multiple perspectives while tracking the SFB using the EMT. From N recorded images, intrinsic and extrinsic camera parameters can be computed. For example, intrinsic parameters can include focal length f, pixel aspect ratio α, center point [u, v], and nonlinear radial lens distortion coefficients κ₁ and κ₂. Extrinsic parameters can include homogeneous transformations [T_{TC}¹, T_{TC}², . . . , T_{TC}^{N}] relating the position and orientation of the SFB relative to the test target. This can be coupled with the corresponding measurements [T_{WS}¹, T_{WS}², . . . , T_{WS}^{N}] relating the sensor to the world reference frame to solve for the unknown transformations T_{SC} and T_{TW} by solving the following system of equations:

$$\begin{matrix}{T}_{TC}^{1} = & {T}_{SC} {T}_{WS}^{1} {T}_{TW} \\ ⋮ & ⋮ \\ {T}_{TC}^{N} = & {T}_{SC} {T}_{WS}^{1 N} {T}_{TW} .\end{matrix}$$

The transformations T_{SC} and T_{TW} can be computed directly from these equations, for example, using singular-value decomposition.

Bronchoscopy

Prior to bronchoscopy, the animal was placed on a flat operating table in the supine position, just above the EMT field generator. An initial registration between the EMT and CT image coordinate systems was performed. FIG. 6C illustrates rigid registration of the EMT system and CT image coordinates, in accordance with many embodiments. The rigid registration can be performed by locating branch-points in the airways of the lung using a tracked stylus inserted into the working channel of a suitable conventional bronchoscope (e.g., an EB-1970K video bronchoscope, Hoya-Pentax). The corresponding landmarks can be located in a virtual surface model of the airways generated by a CT scan as described below, and a point-to-point registration can thus be computed. The SFB and attached EMT sensor can then be placed into the working channel of a conventional bronchoscope for examination. This can be done to provide a means of steering if the SFB is not equipped with tip-bending. Alternatively, if the SFB is equipped with a suitable steering mechanism, it can be used independently of the conventional bronchoscope. During bronchoscopy, the SFB can be extended further into smaller airways beyond the reach of the conventional bronchoscope. Video images can be digitized (e.g., using a Nexeon HD frame grabber from dPict Imaging), and recorded to a workstation at a suitable rate (e.g., approximately 15 frames per second), while the sensor position and pose can be recorded at a suitable rate (e.g., 40.5 Hz). To monitor respiration, EMT sensors can be placed on the animal's abdomen and sternum. FIG. 6D illustrates EMT sensors 504 placed on the abdomen and sternum to monitor respiration, in accordance with many embodiments.

CT Imaging

Following bronchoscopy, the animal was imaged using a suitable CT scanner (e.g., a VCT 64-slice light-speed scanner, General Electric). This can be used to produce volumetric images, for example, at a resolution of 512×512×400 with an isotropic voxel spacing of 0.5 mm. During each scan, the animal can be placed on a continuous positive airway pressure at 22 cm H₂O to prevent respiratory artifacts. Images can be recorded, for example, on digital versatile discs (DVDs), and transferred to a suitable processor or workstation (e.g., a Dell 470 Precision Workstation, 3.40 GhZ CPU, 2 GB RAM) for analysis.

Offline Bronchoscopic Tracking Simulation

The SFB guidance system can be tested using data recorded from bronchoscopy. The test platform can be developed on a processor or workstation (e.g., a workstation as described above, using an ATI FireGL V5100 graphics card and running Windows XP). The software test platform can be developed, for example, in C++ using the Visualization Toolkit or VTK (Kitware) that provides a set of OpenGL-supported libraries for graphical rendering. Before simulating tracking of the bronchoscope, an initial image analysis can be used to crop the lung region of the CT images, perform a multistage airway segmentation algorithm, and apply a contouring filter (e.g., from VTK) to produce a surface model of the airways.

Video Preprocessing

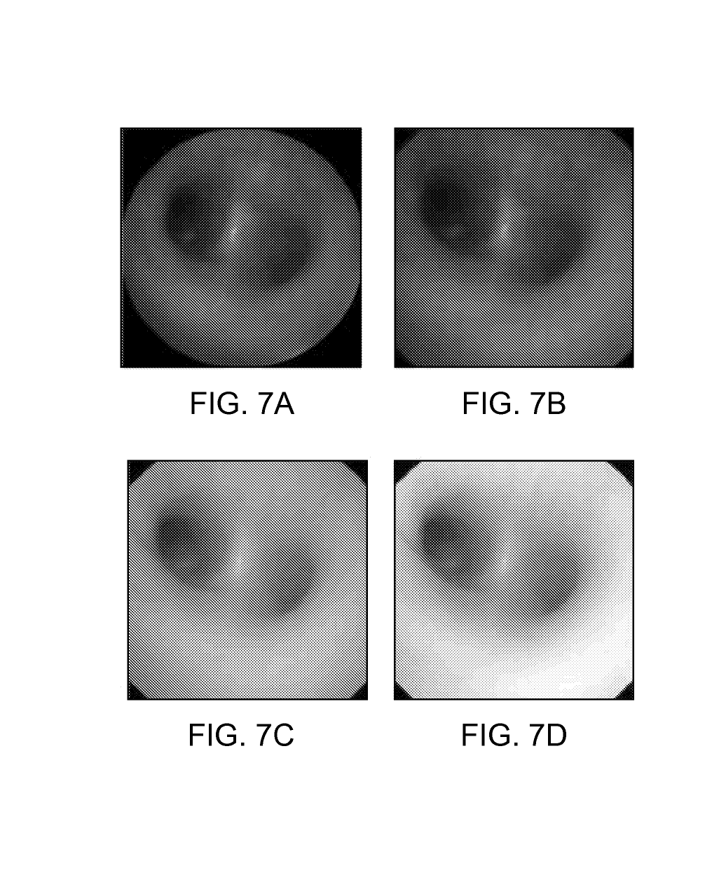

Prior to registration of the SFB video images to the CT-generated virtual model (hereinafter “CT-video registration”), each video image or frame can first be preprocessed. FIG. 7A illustrates correction of radial lens distortion of an image. The correction can be performed, for example, using the intrinsic camera parameters computed as described above. FIG. 7B illustrates conversion of an undistorted color image to grayscale. FIG. 7C illustrates vignetting compensation of an image (e.g., using a vignetting compensation filter) to adjust for the radial-dependent drop in illumination intensity. FIG. 7D illustrates noise removal from an image using a Gaussian smoothing filter.

CT-Video Registration

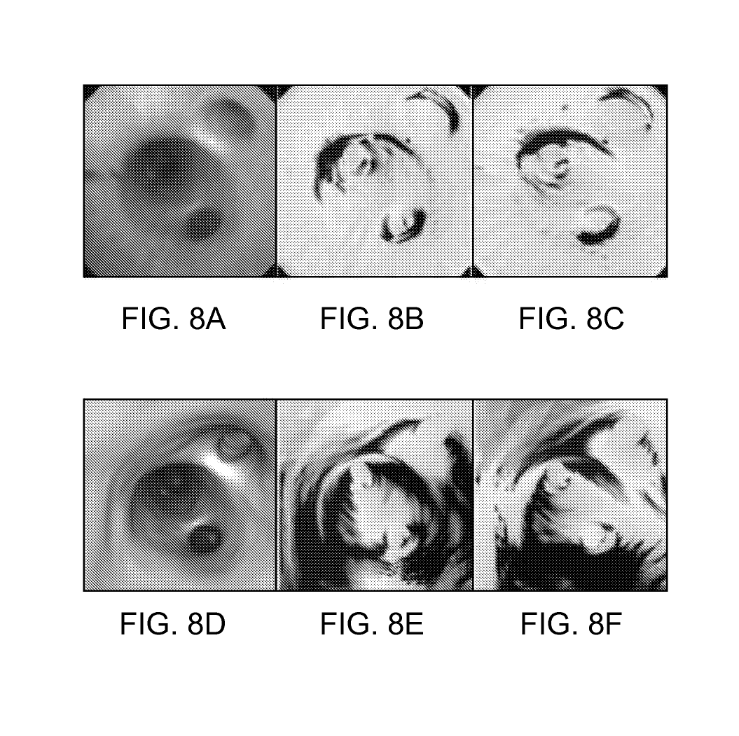

CT-video registration can optimize the position and pose \tilde{x} of the SFB in CT coordinates by maximizing similarity between real and virtual bronchoscopic views, I^{V} and I_{\tilde{x}}^{CT}. Similarity can be measured by differential surface analysis. FIG. 8A illustrates a 2D input video frame I^{V}. The video frame I^{V} can be converted to pq-space, where p and q represent approximations to the 3D surface gradients ∂Z_{C}/∂X_{C} and ∂Z_{C}/∂Y_{C} in camera coordinates, respectively. FIGS. 8B and 8C are vector images defining the p and q gradients, respectively. A gradient image n^{V} can be computed, where each pixel is a 3D gradient vector given by nᵢⱼ^{V}=[pᵢⱼ, qᵢⱼ, −1]. FIG. 8D illustrates a virtual bronchoscopic view obtained from the CT-based reconstruction, C. The surface gradient image n^{CT} from the virtual view can be computed from the 3D geometry of the preexisting surface model, where nᵢⱼ^{CT}=[p′ᵢⱼ, q′ᵢⱼ, −1]. Surface gradients p′ and a′, illustrated in FIGS. 8E and 8F, respectively, can be computed by differentiating the z-buffer of C. Similarity can be measured from the overall alignment of the surface gradients at each pixel as

$$S = \frac{{\sum }_{i = 0}^{N - 1} {\sum }_{j = 0}^{N - 1} {w}_{ij} \cdot {n}_{ij}^{V} \cdot {n}_{ij}^{CT} / ( ‖ {n}_{ij}^{V} ‖ \cdot ‖ {n}_{ij}^{CT} ‖ )}{{\sum }_{i = 0}^{N - 1} {\sum }_{j = 0}^{N - 1} {w}_{ij}} .$$

The weighting term wᵢⱼ can be set equal to the gradient magnitude ∥nᵢⱼ^{V}∥ to permit greater influence from high-gradient regions and improve registration stability. In some instances, limiting the weighting can be necessary, lest similarity be dominated by a very small number of pixels with spuriously large gradients. Accordingly, wᵢⱼ can be set to min(∥nᵢⱼ^{V}∥,10). Optimization of the registration can use any suitable algorithm, such as the constrained, nonlinear, direct, parallel optimization using trust region (CONDOR) algorithm.

Hybrid Tracking

In many embodiments, both EMT and IBT can provide independent estimates of the 6D position and pose \tilde{x}=[x^{T}, θ^{T}]^{T} of the SFB in static CT coordinates, as it navigates through the airways. In the hybrid implementation, the position and pose recorded by the EMT sensor \tilde{x}ₖ^{EMT} can provide an initial estimate of the SFB position and pose at each frame k. This can then be refined to as \tilde{x}ₖ^{CT} by CT-video registration, as described above. The position disagreement between the two tracking sources can be modeled as

xₖ^{CT}=xₖ^{EMT}+δₖ.

If xₖ^{CT} is assumed to be an accurate measure of the true SFB position in the static CT image, δ is the local registration error between the actual and virtual airway anatomies, and can be given by δ=[δₓ; δ_{y}, δ_{z}]^{T}. The model can be expanded to include an orientation term θ, which can be defined as a vector of three Euler angles θ=[θ_{z}, θ_{y}, θ_{z}]^{T}. The relationship of θ to the tracked orientations θ^{EMT} and θ^{CT} can be given by

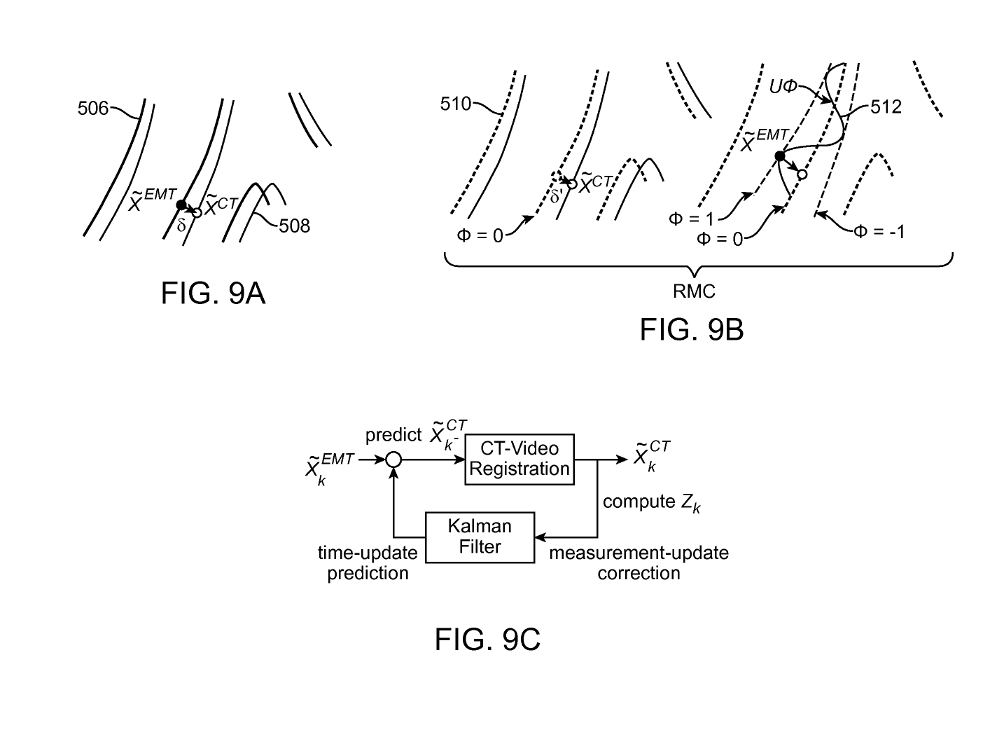

R(θₖ^{CT})=R(θₖ^{EMT})R(θₖ)

where R(θ) is the resulting rotation matrix computed from θ. Both δ and θ can be assumed to vary slowly with time, as illustrated in FIG. 9A (xₖ^{EMT} is trace 506, xₖ^{CT} is trace 508). An error-state Kalman filter can be implemented to adaptively estimate δₖ and θₖ over the course of the bronchoscopy.

Generally, the discrete Kalman filter can be used to estimate the unknown state ŷ of any time-controlled process from a set of noisy and uniformly time-spaced measurements z using a recursive two-step prediction stage and subsequent measurement-update correction stage. At each measurement k, an initial prediction of the Kalman state ŷₖ⁻ can be given by

ŷₖ⁻=Aŷₖ₋₁

Pₖ⁻=APₖ₋₁A^{T}+Q (time-update prediction)

where A is the state transition matrix, P is the estimated error covariance matrix, and Q is the process error covariance matrix. In the second step, the corrected state estimate ŷₖ can be calculated from the measurement zₖ by using

Kₖ=Pₖ⁻H^{T}(HPₖ⁻H^{T}+R)

ŷₖ=ŷₖ⁻+Kₖ(zₖ−ŷₖ⁻)

Pₖ=(I−KₖH)Pₖ⁻ (measurement-update correction)

where K is the Kalman gain matrix, H is the measurement matrix, and R is the measurement error covariance matrix.

From the process definition described above, an error-state Kalman filter can be used to recursively compute the registration error between \tilde{x}^{EMT} and \tilde{x}^{CT} from the error state ŷ=[δₓ, δ_{y}, δ_{z}, θ_{z}, θ_{y}, θ_{z}]^{T}. At each new frame, an improved initial estimate \tilde{x}ₖ^{CT} can be computed from the predicted error state ŷₖ⁻, where A is simply an identity matrix, and the predicted position and pose can be given by xₖ^{CT}=xₖ^{EMT}+δₖ and R (θₖ^{CT})=R(θₖ^{EMT})R(θₖ). Following CT-video registration, the measured error zₖ can be equal to [zₓ^{T}, z_{θ}T]^{T}, where zₓ^{T}=x^{CT}−x^{EMT} and z_{θ} contains the three Euler angles that correspond to the rotational error R(θ^{EMT})⁻¹R(θ^{CT}). A measurement update can be performed as described above. In this way, the Kalman filter can be used to adaptively recomputed updated measurements of δ and θ, which vary with time and position in the airways.

In some instances, however, the aforementioned model can be limited by its assumption that the registration error is slowly varying in time, and can be further refined. When considering the effect of respiratory motion, the registration error can be differentiated into two components: a slowly varying error offset δ′ and an oscillatory component that is dependent on the respiratory phase φ, where φ varies from 1 at full inspiration to −1 at full expiration. Therefore, the model can be extended to include respiratory motion compensation (RMC), given by the form

xₖ^{CT}=xₖ^{EMT}+δ′ₖ+φₖUₖ.

FIG. 9B illustrates RMC in which registration error is differentiated into a zero-phase offset δ′ (indicated by the dashed trace 510 at left) and a higher frequency phase-dependent component Uφ (indicated by trace 512 at right).

In this model, δ′ can represent a slowly varying secular error between the EMT system and the zero-phase or “average” airway shape at φ=0. The process variable Uₖ can be the maximum local deformation between the zero-phase and full inspiration (φ=1) or expiration (φ=−1) at \tilde{x}ₖ^{CT}. Deformable registration of chest CT images taken at various static lung pressure can show that the respiratory-induced deformation of a point in the lung roughly scales linearly with the respiratory phase between full inspiration and full expiration. Instead of computing φ from static lung pressures, an abdominal-mounted position sensor can serve as a surrogate measure of respiratory phase. The abdominal sensor position can be converted to φ by computing the fractional displacement relative to the maximum and minimum displacements observed in the previous two breath cycles. In many embodiments, it is possible to compensate for respiratory-induced motion directly. The original error state vector ŷ can be revised to include an estimation of U, such that ŷ=[δₓ, δ_{y}, δ_{z}, θ_{z}, θ_{y}, θ_{z}, Uₓ, U_{y}, U_{z}]^{T}. The initial position estimate can be modified to: xₖ^{CT}=xₖ^{EMT}+δ′ₖ+φₖUₖ. FIG. 9C is a schematic illustration by way of block diagram illustrating the hybrid tracking algorithm, in accordance with many embodiments of the present invention.

### Example

### Hybrid Tracking Simulation Results

A hybrid tracking simulation is performed as described above. From a total of six bronchoscopic sections, four are selected for analysis. In each session, the SFB begins in the trachea and is progressively extended further into the lung until limited by size or inability to steer. Each session constitutes 600-1000 video frames, or 40-66 s at a 15 Hz frame rate, which provides sufficient time to navigate to a peripheral region. Two sessions are excluded, mainly as a result of mucus, which makes it difficult to maneuver the SFB and obscures images.

Validation of the tracking accuracy is performed by registrations performed manually at a set of key frames, spaced at every 20^{th} frame of each session. Manual registration requires a user to manipulate the position and pose of the virtual camera to qualitatively match the real and virtual bronchoscopic images by hand. The tracking error E^{key} is given as the root mean squared (RMS) positional and orientational error between the manually registered key frames and hybrid tracking output, and is listed in TABLE 1.

TABLE 1
Average statistics for each of the SFB tracking methodologies

| | EMT | IBT | H1 | H2 | H3 |
|---|---|---|---|---|---|
| E^{key} | 14.22 | 14.92 | 6.74 | 4.20 | 3.33 |
| (mm/°) | 18.52° | 51.30° | 14.30° | 11.90° | 10.01° |
| E^{pred} | — | — | 4.82 | 3.92 | 1.96 |
| (mm/°) | | | 18.64° | 9.44° | 8.20° |
| E^{blind} | — | — | 5.12 | 4.17 | 2.73 |
| (mm/°) | | | 22.61° | 17.83° | 16.65° |
| Δx | — | 1.52 | 4.53 | 3.33 | 2.37 |
| (mm/°) | | 7.53° | 10.94° | 10.95° | 8.46° |
| # iter. | — | 109.3 | 157.1 | 138.5 | 121.9 |
| time (s) | — | 1.92 | 2.61 | 2.48 | 2.15 |

Error metrics E^{key}, E^{pred}, E^{blind}, and Δ\tilde{x} are given as RMS position and orientation errors over all frames. The mean number of optimizer iterations and associated execution times are listed for CT-video registration under each approach.

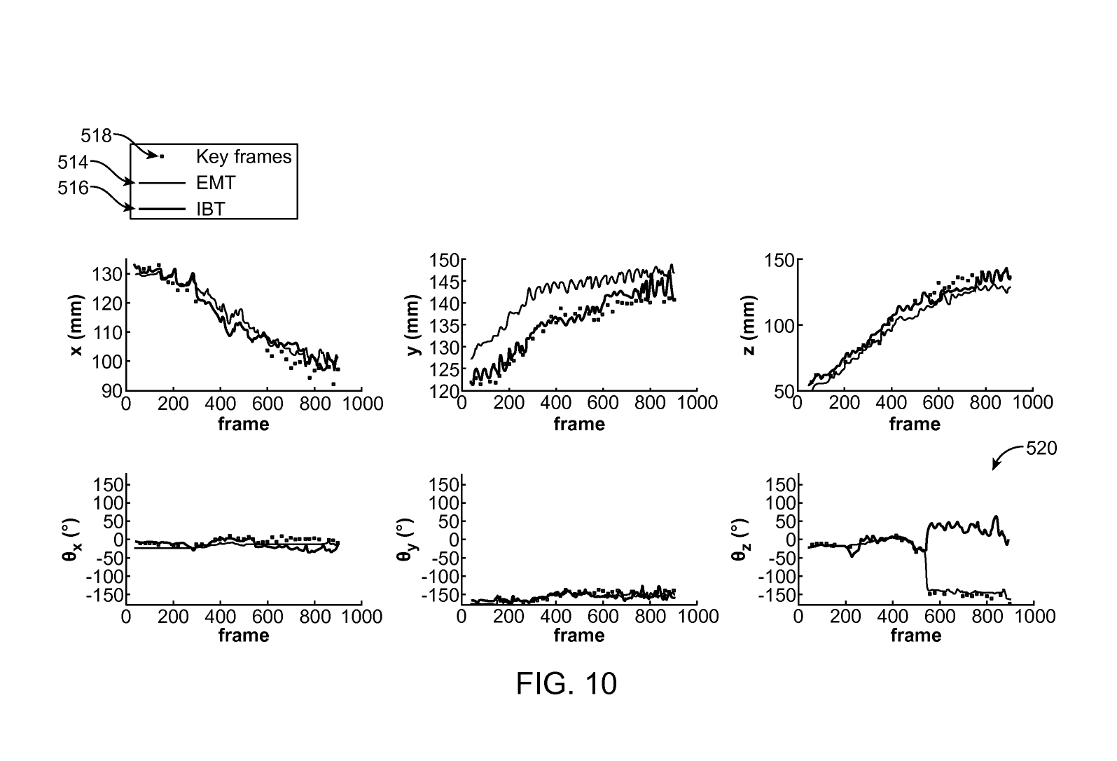

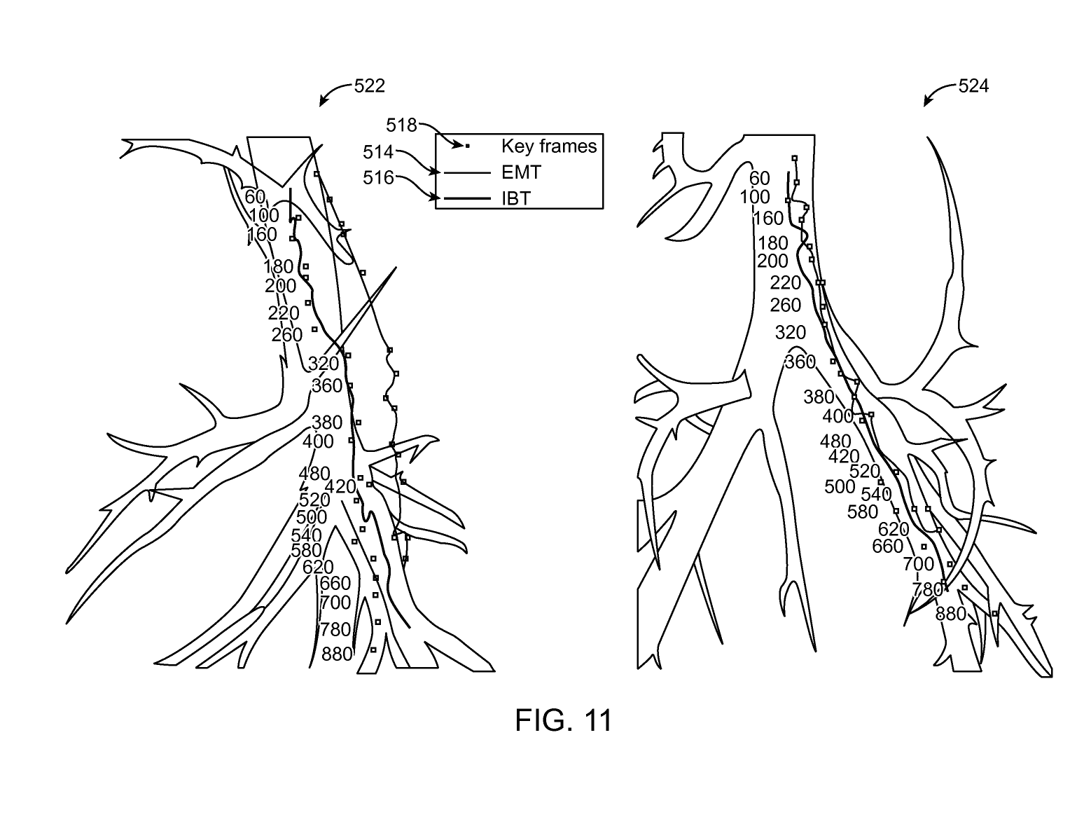

For comparison, tracking is initially performed by independent EMT or IBT. Using just the EMT system, E^{key} is 14.22 mm and 18.52° averaged over all frames. For IBT, E^{key} is 14.92 mm and 52.30° averaged over all frames. While this implies that IBT is highly inaccurate, these error values are heavily influenced by periodic misregistration of real and virtual bronchoscopic images, causing IBT to deviate from the true path of the SFB. As such, IBT alone is insufficient for reliably tracking the SFB into peripheral airway regions. FIGS. 10 and 11 depict the tracking results from independent EMT and IBT over the course of session 1 relative to the recorded frame number. In FIG. 10, tracked position and orientation of the SFB using EMT (represented by traces 514) and IBT (represented by traces 516) are plotted against the manually registered key frames (represented by dots 518) in each dimension separately. EMT appears fairly robust, though small registration errors prevent adequate localization, especially within the smaller airways. By contrast, IBT can accurately reproduce motion of the SFB, though misregistration causes tracking to diverge from the true SFB path. As evident from the plot 520 of θ_{z} in FIG. 10, the SFB is twisted rather abruptly at around frame 550, causing a severe change in orientation that cannot be recovered by CT-video registration. In FIG. 11, tracking results from session 1 are subsampled and plotted as 3D paths within the virtual airway model along with the frame number. This path is depicted from the sagittal view 522 and coronal view 524. Due to misregistration between real and virtual anatomies, localization by EMT contains a high degree of error. Using IBT, accurate localization is achieved until near the end of the session, where it fails to recognize that the SFB has accessed a smaller side branch shown at key frame 880.

Hybrid Tracking

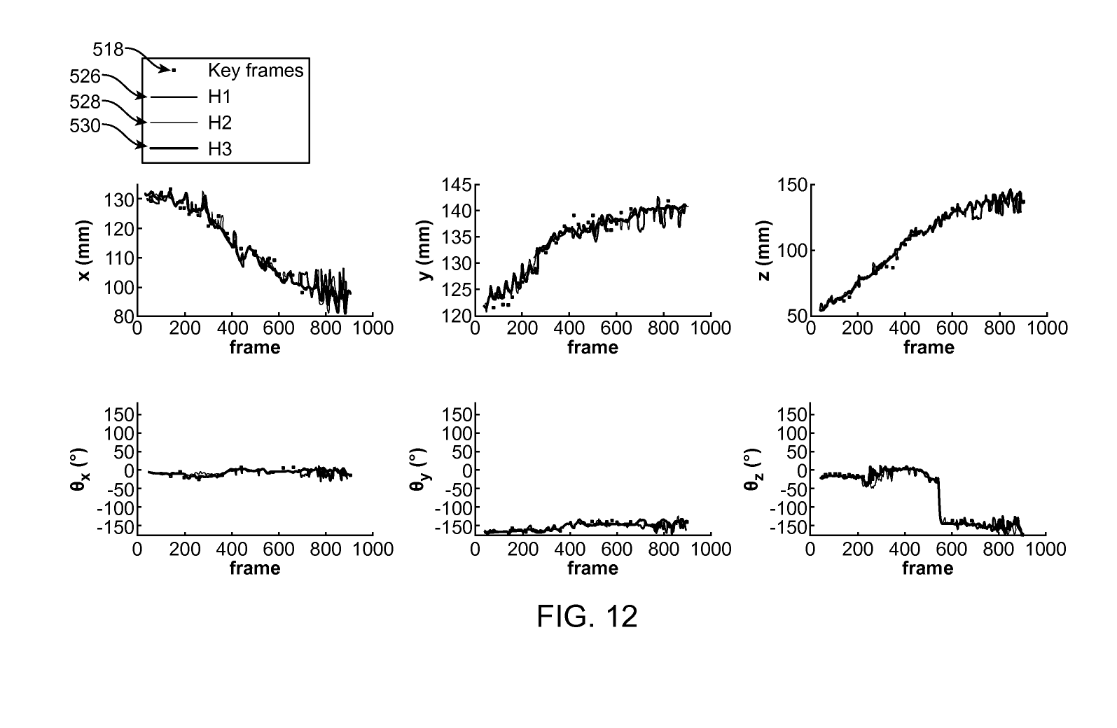

Three hybrid tracking methods are compared for each of the four bronchoscopic sessions. In the first hybrid method (H1), only the registration error δ is considered. In the second method (H2), the orientation correction term θ is added. In the third method (H3), RMC is further added, differentiating the tracked position discrepancy of EMT and IBT into a relative constant δ′ and a respiratory motion-dependent term φU. The positional tracking error E^{key} is 6.74, 4.20, and 3.33 mm for H1, H2, and H3, respectively. The orientational error E_{θ}^{key} is 14.30°, 11.90°, and 10.01° for H1, H2, and H3, respectively. FIG. 12 depicts the tracking accuracy for each of the methods in session 1 relative to the key frames 518. Hybrid tracking results from session 1 are plotted using position only (H1, depicted as traces 526), plus orientation (H2, depicted as traces 528), and finally, with RMC (H3 depicted as traces 530) versus the manually registered key frames. Each of the hybrid tracking methodologies manages to follow the actual course; however, addition of orientation and RMC into the hybrid tracking model greatly stabilize localization. This is especially apparent at the end of the plotted course where the SFB has accessed more peripheral airways that undergo significant respiratory-induced displacement. Though all three methods track the same general path, H1 and H2 exhibit greater noise. Tracking noise is quantified by computing the average interframe motion Ox between subsequent localizations at \tilde{x}ₖ₋₁^{CT} and \tilde{x}ₖ^{CT}. Average interframe motion Ox is 4.53 mm and 10.94° for H1, 3.33 mm and 10.95° for H2, and 2.37 mm and 8.46° for H3.

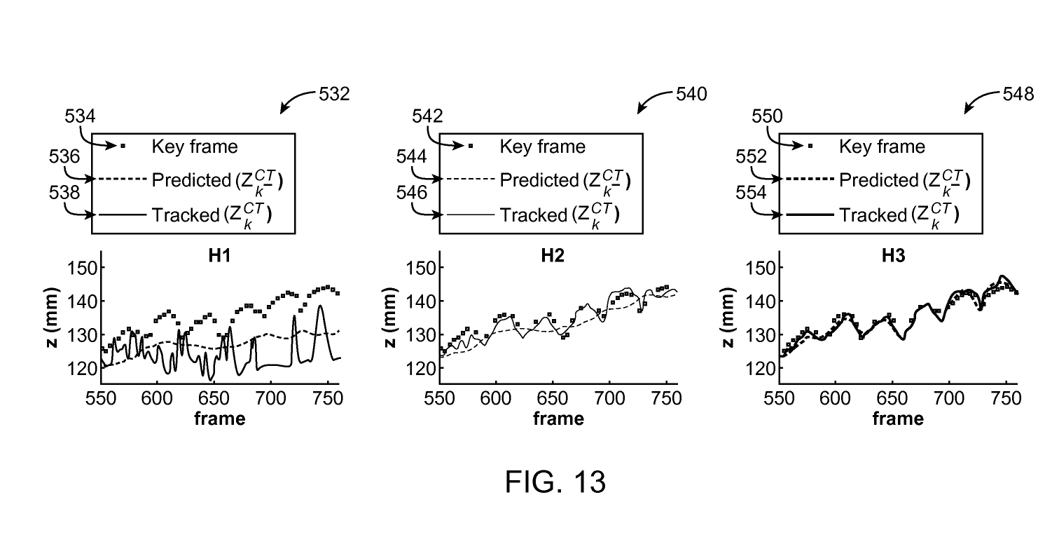

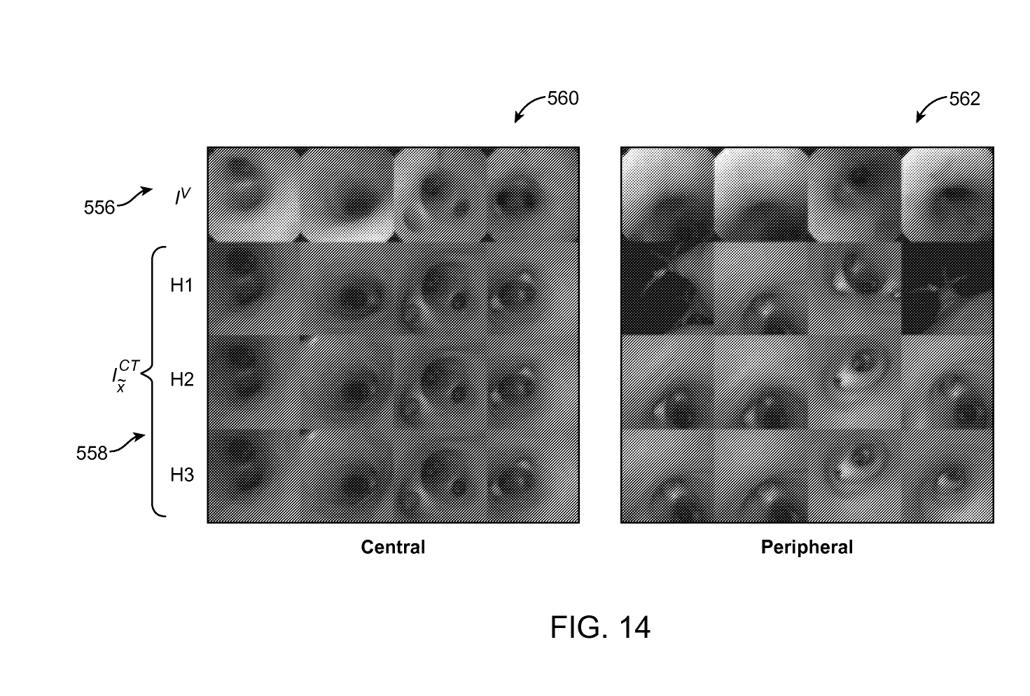

To eliminate the subjectivity inherent in manual registration, prediction error E^{pred} is computed as the average per-frame error between the predicted position and pose, \tilde{x}ₖ^{CT}, and tracked position \tilde{x}ₖ^{CT}. The position prediction error Eₓ^{pred} is 4.82, 3.92, and 1.96 mm for methods H1, H2, and H3, respectively. The orientational prediction error E_{θ}^{pred} is 18.64°, 9.44°, and 8.20° for H1, H2, and H3, respectively. FIG. 13 depicts the z-axis tracking results for each of the hybrid methods within a peripheral region of session 4. For each plot, the tracked position is compared to the predicted position and key frames spaced every four frames. Key frames (indicated by dots 534, 542, 550) are manually registered at four frame intervals. For each method, the predicted z position zₖ₋^{CT} (indicated by traces 536, 544, 552) is plotted along with the tracked position zₖ₋^{CT} (indicated by traces 538, 546, 554). In method H1 (depicted in plot 532), prediction error results in divergent tracking. In method H2 (depicted in plot 540), the addition of orientation improves tracking accuracy, although prediction error is still large, as 6 does not react quickly to the positional error introduced by respiration. In method H3 (depicted in plot 548), the tracking accuracy is modestly improved, though the predicted position more closely follows the tracked motion. The z-component is selected because it is the axis along which motion is most predominant. FIG. 14 shows registered real bronchoscopic views 556 and virtual bronchoscopic views 558 at selected frames using all three methods. Tracking accuracy is somewhat more comparable in the central airways, as represented by the left four frames 560. In the more peripheral airways (right four frames 562), the positional offset model cannot reconcile the prediction error, resulting in frames that fall outside the airways altogether. Once orientation is added, tracking stabilizes, though respiratory motion at full inspiration or expiration is observed to cause misregistration. With RMC, smaller prediction errors result in more accurate tracking.

From the proposed hybrid models, the error terms in ŷ are considered to be locally consistent and physically meaningful, suggesting that these values are not expected to change dramatically over a small change in position. Provided this is true, \tilde{x}ₖ^{CT} at each frame should be relatively consistent with a blind prediction of the SFB position and pose computed from ŷ_{k-τ}, at some small time in the past. Formally, the blind prediction error for position Eₓ^{blind} can be computed as

$$\begin{matrix}{E}_{{x}_{k}}^{blind} ( τ ) = \{ \begin{matrix}‖ {x}_{k}^{CT} - ( {x}_{k}^{EMT} + {δ}_{k - τ} ) ‖ \\ ‖ {x}_{k}^{CT} - ( {x}_{k}^{EMT} + {δ}_{k - τ}^{′} + {φ}_{k} {U}_{k - τ} ) ‖\end{matrix} . & H1 \\ & H 3\end{matrix}$$

For time, a time lapse of τ−1 s, Eₓ_{ₖ}^{blind} is 4.53, 3.33, and 2.37 mm for H1, H2, and H3, respectively.

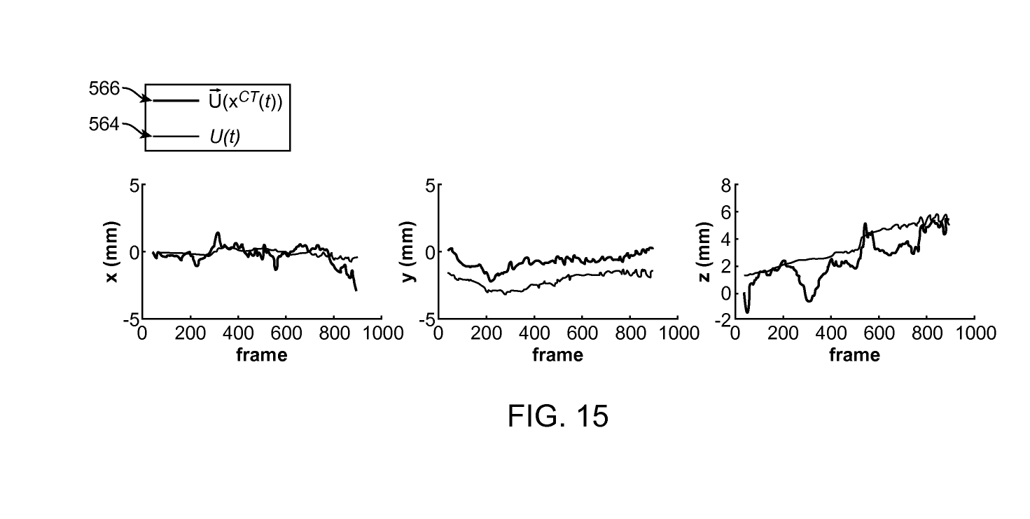

From the hybrid model H3, RMC produces an estimate of the local and position-dependent airway deformation U=U(x^{CT}). Unlike the secular position and orientation errors, δ and θ, U is assumed to be a physiological measurement, and therefore, it is independent of the registration. For comparison, the computed deformation is also independently measured through deformable image registration of two CT images taken at full inspiration and full expiration (lung pressures of 22 and 6 cm H₂O, respectively). From this process, a 3D deformation field \vec{U} is calculated, describing the maximum displacement of each part of the lung during respiration. FIG. 15 compares the maximum deformation approximated by the Kalman filter U(x^{CT}) over every frame of the first bronchoscopic session to that calculated from the deformation field \vec{U}(x^{CT}).) The deformation U (traces 564), computed from the hybrid tracking algorithm using RMC, is compared to the deformation \vec{U}(x^{CT}) (traces 566), computed from non-rigid registration of two CT images at full inspiration and full expiration. The maximum displacement values at each frame Uₖ and \vec{U}ₖ represent the respiratory-induced motion of the airways at each point in the tracked path x^{CT} from the trachea to the peripheral airways. As evident from the graphs, deformation is most predominant in the z-axis and in peripheral airways, where displacements of ±5 mm z-axis are observed.

The results show that the hybrid approach provides a more stable and accurate means of localizing the SFB intraoperatively. The positional tracking error E^{key} for EMT and IBT is 14.22 and 14.92 mm, respectively, as compared to 6.74 mm in the simplest hybrid approach. Moreover, Eₓ^{key} reduces by at least two-fold from the addition of orientation and RMC to the process model. After introducing the rotational correction, the predicted orientation error E_{θ}^{key} reduces from 18.64° to 9.44°. Likewise, RMC reduces the predicted position error Eₓ^{pred} from 3.92 to 1.96 mm and the blind prediction error Eₓ^{blind} from 4.17 mm to 2.73 mm.

Using RMC, the Kalman error model more accurately predicts SFB motion, particularly in peripheral lung regions that are subject to large respiratory excursions. From FIG. 15, the maximum deformation U estimated by the Kalman filter is around ±5 mm in the z-axis, or 10 mm in total, which agrees well with the deformation computed from non-rigid registration of CT images at full inspiration and full expiration.

Overall, the results from in vivo bronchoscopy of peripheral airways within a live, breathing pig are promising, suggesting that image-guided TBB may be clinically viable for small peripheral pulmonary nodules.

Virtual Surgical Field

Suitable embodiments of the systems, methods, and devices for endoscope tracking described herein can be used to generate a virtual model of an internal structure of the body. In many embodiments, the virtual model can be a stereo reconstruction of a surgical site including one or more of tissues, organs, or surgical instruments. Advantageously, the virtual model as described herein can provide a 3D model that is viewable from a plurality of perspectives to aid in the navigation of surgical instruments within anatomically complex sites.

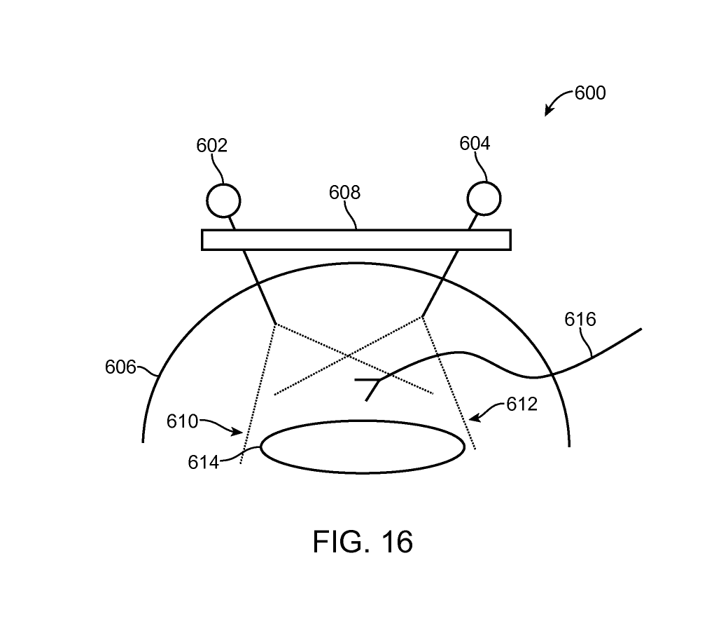

FIG. 16 illustrates an endoscopic system 600, in accordance with many embodiments. The endoscopic system 600 includes a plurality of endoscopes 602, 604 inserted within the body of a patient 606. The endoscopes 602, 604 can be supported and/or repositioned by a holding device 608, a surgeon, one or more robotic arms, or suitable combinations thereof. The respective viewing fields 610, 612 of the endoscopes 602, 604 can be used to image one or more internal structures with the body, such as a tissue or organ 614, or surgical instrument 616.

Any suitable number of endoscopes can be used in the system 600, such as a single endoscope, a pair of endoscopes, or multiple endoscopes. The endoscopes can be flexible endoscopes or rigid endoscopes. In many embodiments, the endoscopes can be ultrathin fiber-scanning endoscopes, as described herein. For example, one or more ultrathin rigid endoscopes, also known as needle scopes, can be used.

In many embodiments, the endoscopes 602, 604 are disposed relative to each other such that the respective viewing fields or viewpoints 610, 612 are different. Accordingly, a 3D virtual model of the internal structure can be generated based on image data captured with respect to a plurality of different camera viewpoints. For example, the virtual model can be a surface model representative of the topography of the internal structure, such as a surface grid, point cloud, or mosaicked surface. In many embodiments, the virtual model can be a stereo reconstruction of the structure generated from the image data (e.g., computed from disparity images of the image data). The virtual model can be presented on a suitable display unit (e.g., a monitor, terminal, or touchscreen) to assist a surgeon during a surgical procedure by providing visual guidance for maneuvering a surgical instrument within the surgical site. In many embodiments, the virtual model can be translated, rotated, and/or zoomed to provide a virtual field of view different than the viewpoints provided by the endoscopes. Advantageously, this approach enables the surgeon to view the surgical site from a stable, wide field of view even in situations when the viewpoints of the endoscopes are moving, obscured, or relatively narrow.

In order to generate a virtual model from a plurality of endoscopic viewpoints, the spatial disposition of the distal image gathering portions of the endoscopes 602, 604 can be determined using any suitable endoscope tracking method, such as the embodiments described herein. Based on the spatial disposition information, the image data from the plurality of endoscopic viewpoints can be aligned to each other and with respect to a global reference frame in order to reconstruct the 3D structure (e.g., using a suitable processing unit or workstation). In many embodiments, each of the plurality of endoscopes can include a sensor coupled to the distal image gathering portion of the endoscope. The sensor can be an EMT sensor configured to track motion with respect to fewer than six DoF (e.g., five DoF), and the full six DoF motion can be determined based on the sensor tracking data and supplemental data of motion, as previously described. In many embodiments, the hybrid tracking approaches described herein can be used to track the endoscopes.

Optionally, the endoscopes 602, 604 can include at least one needle scope having a proximal portion extending outside the body, such that the spatial disposition of the distal image gathering portion of the needle scope can be determined by tracking the spatial disposition of the proximal portion. For example, the proximal portion can be tracked using EMT sensors as described herein, a coupled inertial sensor, an external camera configured to image the proximal portion or a marker on the proximal portion, or suitable combinations thereof. In many embodiments, the needle scope can be manipulated by a robotic arm, such that the spatial disposition of the proximal portion can be determined based on the spatial disposition of the robotic arm.

In many embodiments, the virtual model can registered to a second virtual model. Both virtual models can thus be simultaneously displayed to the surgeon. The second virtual model can be generated based on data obtained from a suitable imaging modality different from the endoscopes, such as one or more of CT, MRI, PET, fluoroscopy, or ultrasound (e.g., obtained during a pre-operative procedure). The second virtual model can include the same internal structure imaged by the endoscopes and/or a different internal structure. Optionally, the internal structure of the second virtual model can include subsurface features relative to the virtual model, such as subsurface features not visible from the endoscopic viewpoints. For example, the first virtual model (e.g., as generated from the endoscopic views) can be a surface model of an organ, and the second virtual model can be a model of one or more internal structures of the organ. This approach can be used to provide visual guidance to a surgeon for maneuvering surgical instruments within regions that are not endoscopically apparent or otherwise obscured from the viewpoint of the endoscopes.

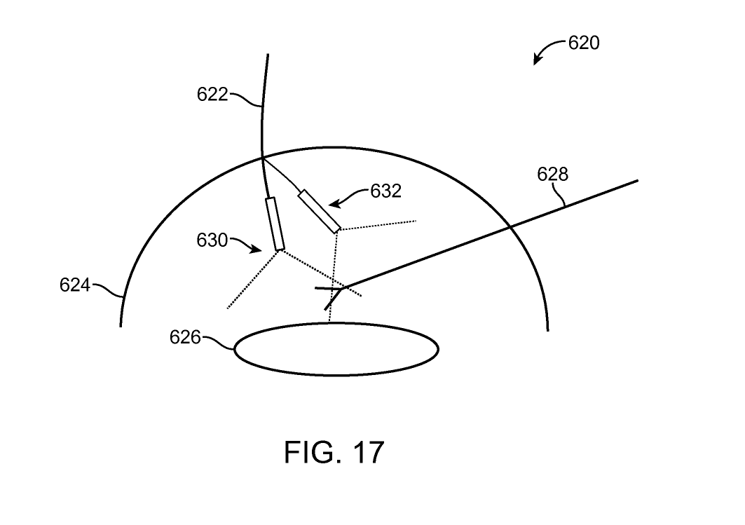

FIG. 17 illustrates an endoscopic system 620, in accordance with many embodiments. The system 620 includes an endoscope 622 inserted within a body 624 and used to image a tissue or organ 626 and surgical instrument 628. Any suitable endoscope can be used for the endoscope 622, such as the embodiments disclosed herein. The endoscope 622 can be repositioned to a plurality of spatial dispositions within the body, such as from a first spatial disposition 630 to a second spatial disposition 632, in order to generate image data with respect to a plurality of camera viewpoints. The distal image gathering portion of the endoscope 622 can be tracked as described herein to determine its spatial disposition. Accordingly, a virtual model can be generated based on the image data from a plurality of viewpoints and the spatial disposition information, as previously described.

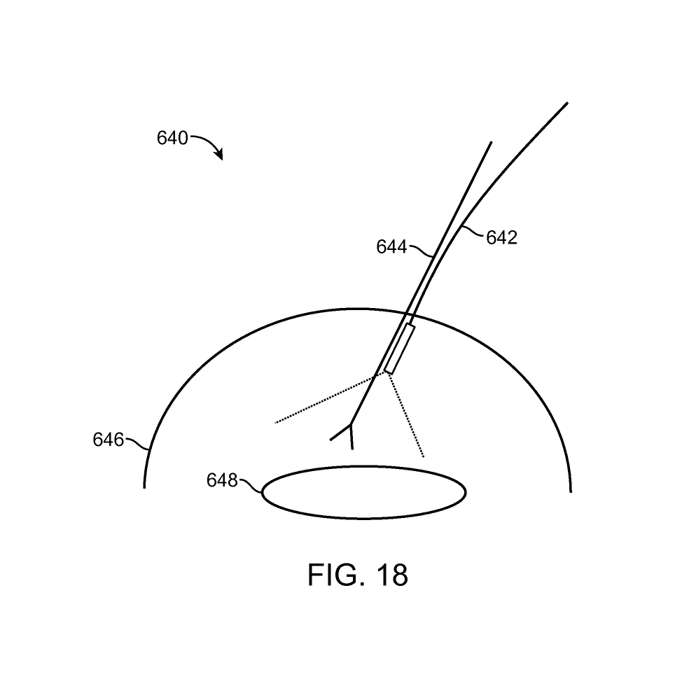

FIG. 18 illustrates an endoscopic system 640, in accordance with many embodiments. The system 640 includes an endoscope 642 coupled to a surgical instrument 644 inserted within a body 646. The endoscope 642 can be used to image the distal end of the surgical instrument 644 as well as a tissue or organ 648. Any suitable endoscope can be used for the endoscope 642, such as the embodiments disclosed herein. The coupling of the endoscope 642 and the surgical instrument 644 advantageously allows both devices to be introduced into the body 646 through a single incision or opening. In some instances, however, the viewpoint provided by the endoscope 642 can be obscured or unstable due to, for example, motion of the coupled instrument 644. Additionally, the co-alignment of the endoscope 642 and the surgical instrument 644 can make it difficult to visually judge the distance between the instrument tip and the tissue surface.

Accordingly, a virtual model of the surgical site can be displayed to the surgeon such that a stable and wide field of view is available even if the current viewpoint of the endoscope 642 is obscured or otherwise less than ideal. For example, the distal image gathering portion of the endoscope 642 can be tracked as previously described to determine its spatial disposition. Thus, as the instrument 644 and endoscope 642 are moved through a plurality of spatial dispositions within the body 646, the plurality of image data generated by the endoscope 642 can be processed, in combination with the spatial disposition information, to produce a virtual model as described herein.

One of skill in the art will appreciate that elements of the endoscopic viewing systems 600, 620, and 640 can be combined in many ways suitable for generating a virtual model of an internal structure. Any suitable number and type of endoscopes can be used for any of the aforementioned systems. One or more of the endoscopes of any of the aforementioned systems can be coupled to a surgical instrument. The aforementioned systems can be used to generate image data with respect to a plurality of camera viewpoints by having a plurality of endoscopes positioned to provide different camera viewpoints, moving one or more endoscopes through a plurality of spatial dispositions corresponding to a plurality of camera viewpoints, or suitable combinations thereof

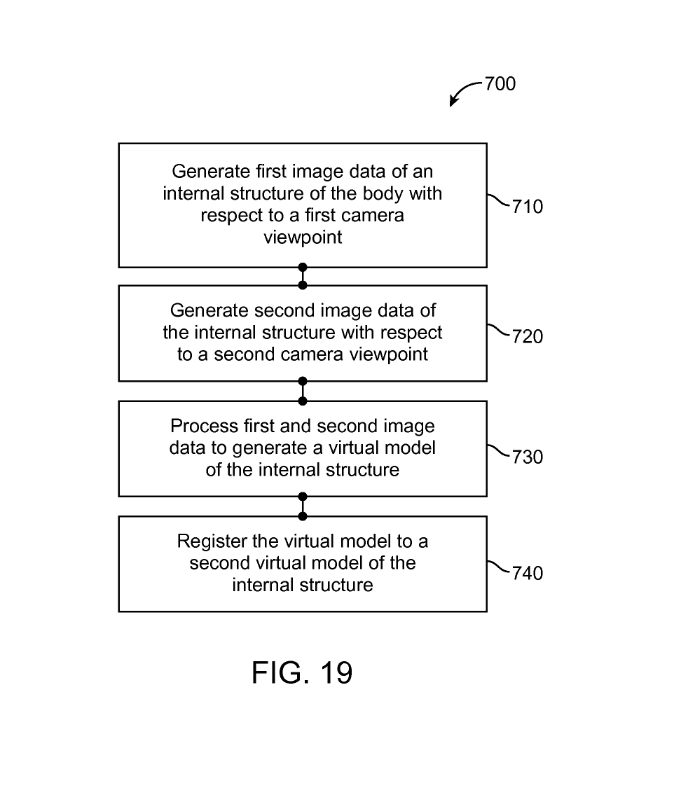

FIG. 19 is a block diagram illustrating acts of a method 700 for generating a virtual model of an internal structure of a body, in accordance with many embodiments. Any suitable system or device can be used to practice the method 700, such as the embodiments described herein.

In act 710, first image data of the internal structure of the body is generated with respect to a first camera viewpoint. The first image data can be generated, for example, with any endoscope suitable for the systems 600, 620, or 640. The endoscope can be positioned at a first spatial disposition to produce image data with respect to a first camera viewpoint. In many embodiments, the image gathering portion of the endoscope can be tracked in order to determine the spatial disposition corresponding to the image data. For example, the tracking can be performed using a sensor coupled to the image gathering portion of the endoscope (e.g., an EMT sensor detecting less than six DoF of motion) and supplemental data of motion (e.g., EMT sensor data and/or image data), as described herein.

In act 720, second image data of the internal structure of the body is generated with respect to a second camera viewpoint, the second camera viewpoint being different than the first. The second image data can be generated, for example, with any endoscope suitable for the systems 600, 620, or 640. The endoscope of act 720 can be the same endoscope used to practice act 710, or a different endoscope. The endoscope can be positioned at a second spatial disposition to produce image data with respect to a second camera viewpoint. The image gathering portion of the endoscope can be tracked in order to determine the spatial disposition, as previously described with regards to the act 710.

In act 730, the first and second image data are processed to generate a virtual model of the internal structure. Any suitable device can be used to perform the act 730, such as the workstation 56. For example, the workstation 56 can include a tangible computer-readable storage medium storing suitable non-transitory instructions that can be executed by one or more processors of the workstation 56 to process the image data. The resultant virtual model can be displayed to the surgeon as described herein (e.g., on a monitor of the workstation 56 or the display unit 62).

In act 740, the virtual model is registered to a second virtual model of the internal structure. The second virtual model can be a provided based on data obtained from a suitable imaging modality (e.g., CT, PET, MRI, fluoroscopy, ultrasound). The registration can be performed by a suitable device, such as the workstation 56, using a tangible computer-readable storage medium storing suitable non-transitory instructions that can be executed by one or more processors to register the models to each other. Any suitable method can be used to perform the model registration, such as a surface matching algorithm. Both virtual models can be presented, separately or overlaid, on a suitable display unit (e.g., a monitor of the workstation 56 or the display unit 62) to enable, for example, visualization of subsurface features of an internal structure.

The acts of the method 700 can be performed in any suitable combination and order. In many embodiments, the act 740 is optional and can be excluded from the method 700. Suitable acts of the method 700 can be performed more than once. For example, during a surgical procedure, the acts 710, 720, 730, and/or 740 can be repeated any suitable number of times in order to update the virtual model (e.g., to provide higher resolution image data generated by moving an endoscope closer to the structure, to display changes to a tissue or organ effected by the surgical instrument, or to incorporate additional image data from an additional camera viewpoint). The updates can occur automatically (e.g., at specified time intervals) and/or can occur based on user commands (e.g., commands input to the workstation 56).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for imaging an internal tissue of a body, the method comprising:
inserting an image gathering portion of a flexible endoscope into the body, the image gathering portion coupled to a sensor configured to sense motion of the image gathering portion with respect to fewer than six degrees of freedom;
generating a tracking signal indicative of the motion of the image gathering portion using the sensor; and
processing the tracking signal in conjunction with supplemental data of the motion of the image gathering portion to determine a spatial disposition of the image gathering portion within the body.

2. (canceled)

3. The method of claim 1, wherein the sensor is configured to sense the motion of the image gathering portion with respect to five degrees of freedom.

4. The method of claim 1, wherein the sensor comprises an electromagnetic tracking sensor.

5. The method of claim 4, wherein the electromagnetic tracking sensor comprises an annular sensor disposed around the image gathering portion.

6.-8. (canceled)

9. The method of claim 1, wherein the supplemental data comprises one or more images collected by the image gathering portion.

10. The method of claim 9, wherein the supplemental data further comprises a virtual model of the body to which the one or more images can be registered.

11. The method of claim 1, wherein processing the tracking signal in conjunction with the supplemental data of the motion of the image gathering portion to determine a spatial disposition of the image gathering portion within the body comprises adjusting for tracking errors caused by motion of the body due to a body function.

12. A system for imaging an internal tissue of a body, the system comprising:
a flexible endoscope comprising an image gathering portion;
a sensor coupled to the image gathering portion, the sensor configured to generate a tracking signal indicative of motion of the image gathering portion with respect to fewer than six degrees of freedom;
one or more processors; and
a tangible storage medium storing non-transitory instructions that, when executed by the one or more processors, process the tracking signal in conjunction with supplemental data of the motion of the image gathering portion to determine a spatial disposition of the image gathering portion within the body.

13. The system of claim 12, wherein the image gathering portion comprises a cantilevered optical fiber configured to scan light onto the internal tissue and a light sensor configured to receive light returning from the internal tissue so as to generate an output signal that can be processed to provide one or more images of the internal tissue.

14. The system of claim 12, wherein the image gathering portion comprises an outer diameter of less than or equal to 2 mm.

15. The system of claim 12, wherein the image gathering portion comprises an outer diameter of less than or equal to 1.6 mm.

16. (canceled)

17. (canceled)

18. The system of claim 12, wherein the flexible endoscope comprises a steering mechanism configured to guide the image gathering portion within the body.

19. The system of claim 12, wherein the sensor is configured to sense the motion of the image gathering portion with respect to five degrees of freedom.

20. The system of claim 12, wherein the sensor comprises an electromagnetic tracking sensor.

21. The system of claim 20, wherein the electromagnetic tracking sensor comprises an annular sensor disposed around the image gathering portion.

22.-24. (canceled)

25. The system of claim 12, wherein the supplemental data of the motion comprises one or more images collected by the image gathering portion.

26. The system of claim 25, wherein the supplemental data of the motion further comprises a virtual model of the body to which the one or more images can be registered.

27. The system of claim 12, instructions, when executed by the one or more processors, process the tracking signal in conjunction with the supplemental data of the motion of the image gathering portion to determine a spatial disposition of the image gathering portion within the body while adjusting for tracking errors caused by motion of the body due to a body function.

28.-51. (canceled)

52. The method of claim 1, wherein the sensor comprises a single five degree of freedom sensor and the processing comprises recovering a missing degree of freedom based on the supplemental data of the motion.

53. The system of claim 12, wherein the flexible endoscope comprises a single five degree of freedom sensor.
